SEQUENCE LISTING (27 sequences)

SEQ ID NO: 1 - protein, 277 aa, Homo sapiens
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPASKPAGDQLPAALWTSSAVLGLLLLALPTYHLWKRCRHLAEDDTHPPASLRLLPQVSAWAGLRGTGQVGISPS

SEQ ID NO: 2 - protein, 364 aa, Homo sapiens
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 3 - protein, 364 aa, Homo sapiens; identical to SEQ ID NO: 2

SEQ ID NO: 4 - protein, 388 aa, Homo sapiens
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPEPPNTTSPESPDTTSPESPDTTSQEPPDTTSQEPPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 5 - protein, 388 aa, Homo sapiens; identical to SEQ ID NO: 4

SEQ ID NO: 6 - protein, 364 aa, Homo sapiens
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSPEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 7 - protein, 364 aa, Homo sapiens; identical to SEQ ID NO: 2

SEQ ID NO: 8 - protein, 999 aa, Homo sapiens
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFTIVIISSSLLLGLIVLLLISYVMWKAGFFKRQYKSILQEENRRDSWSYINSKSNDD

SEQ ID NO: 9 - protein, 779 aa, Homo sapiens
ELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 10 - protein, 385 aa, Homo sapiens
MGCRRTREGPSKAMIFPWKCQSTQRDLWNIFKLWGWTMLCCDFLAHHGTDCWTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYNPLFIPVAVMVTAFSGLAFIIWLARRLKKGKKSKRSMNDPY

SEQ ID NO: 11 - protein, 382 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 12 - protein, 1032 aa, Homo sapiens [sequence omitted]

SEQ ID NO: 13 - protein, 798 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 14 - DNA, 1546 bp, Homo sapiens
gggactgagcatggatttcggactggccctcctgctggcggggcttctggggctcctcctcggccagtccctccaggtgaagcccctgcaggtggagcccccggagccggtggtggccgtggccttgggcgcctcgcgccagctcacctgccgcctggcctgcgcggaccgcggggcctcggtgcagtggcggggcctggacaccagcctgggcgcggtgcagtcggacacgggccgcagcgtcctcaccgtgcgcaacgcctcgctgtcggcggccgggacccgcgtgtgcgtgggctcctgcgggggccgcaccttccagcacaccgtgcagctccttgtgtacgccttcccggaccagctgaccgtctccccagcagccctggtgcctggtgacccggaggtggcctgtacggcccacaaagtcacgcccgtggaccccaacgcgctctccttctccctgctcgtcgggggccaggaactggagggggcgcaagccctgggcccggaggtgcaggaggaggaggaggagccccagggggacgaggacgtgctgttcagggtgacagagcgctggcggctgccgcccctggggacccctgtcccgcccgccctctactgccaggccacgatgaggctgcctggcttggagctcagccaccgccaggccatccccgtcctgcacagcccgacctccccggagcctcccgacaccacctccccggagtctcccgacaccacctccccggagtctcccgacaccacctcccaggagcctcccgacaccacctccccggagcctcccgacaagacctccccggagcccgccccccagcagggctccacacacacccccaggagcccaggctccaccaggactcgccgccctgagatctcccaggctgggcccacgcagggagaagtgatcccaacaggctcgtccaaacctgcgggtgaccagctgcccgcggctctgtggaccagcagtgcggtgctgggactgctgctcctggccttgcccacctatcacctctggaaacgctgccggcacctggctgaggacgacacccacccaccagcttctctgaggcttctgccccaggtgtcggcctgggctgggttaagggggaccggccaggtcgggatcagcccctcctgagtggccagcctttccccctgtgaaagcaaaatagcttggaccccttcaagttgagaactggtcagggcaaacctgcctcccattctactcaaagtcatccctctgttcacagagatggatgcatgttctgattgcctctttggagaagctcatcagaaactcaaaagaaggccactgtttgtctcacctacccatgacctgaagcccctccctgagtggtccccacctttctggacggaaccacgtactttttacatacattgattcatgtctcacgtctccctaaaaatgcgtaagaccaagctgtgccctgaccaccctgggcccctgtcgtcaggacctcctgaggctttggcaaataaacctcctaaaatgataaaaaaaaaa

SEQ ID NO: 15 - DNA, 2442 bp, Homo sapiens
aggaggaaggggagggaaaaggggaggaggaggaggatgtgagactgggttagagaaatgaaagaaagcaaggctttctgttgacattcagtgcagtctacctgcagcacagcacactccctttgggcaaggacctgagacccttgtgctaagtcaagaggctcaatgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaaatcgcccttggtgaaagaaaattcttggaatactaaaaatcatgagatcctttaaatccttccatgaaacgttttgtgtggtggcacctcctacgtcaaacatgaagtgtgtttccttcagtgcatctgggaagatttctacctgaccaacagttccttcagcttccatttcgcccctcatttatccctcaacccccagcccacaggtgtttatacagctcagctttttgtcttttctgaggagaaacaaataagaccataaagggaaaggattcatgtggaatataaagatggctgactttgctctttcttgactcttgttttcagtttcaattcagtgctgtacttgatgacagacacttctaaatgaagtgcaaatttgatacatatgtgaatatggactcagttttcttgcagatcaaatttcacgtcgtcttctgtatactgtggaggtacactcttatagaaagttcaaaaagtctacgctctcctttctttctaactccagtgaagtaatggggtcctgctcaagttgaaagagtcctatttgcactgtagcctcgccgtctgtgaattggaccatcctatttaactggcttcagcctccccaccttcttcagccacctctctttttcagttggctgacttccacacctagcatctcatgagtgccaagcaaaaggagagaagagagaaatagcctgcgctgttttttagtttgggggttttgctgtttccttttatgagacccattcctatttcttatagtcaatgtttcttttatcacgatattattagtaagaaaacatcactgaaatgctagctgcaagtgacatctctttgatgtcatatggaagagttaaaacaggtggagaaattccttgattcacaatgaaatgctctcctttcccctgcccccagaccttttatccacttacctagattctacatattctttaaatttcatctcaggcctccctcaaccccaccacttcttttataactagtcctttactaatccaacccatgatgagctcctcttcctggcttcttactgaaaggttaccctgtaacatgcaattttgcatttgaataaagcctgctttttaagtgttaact

SEQ ID NO: 16 - DNA, 2798 bp, Homo sapiens
cgttgctgtcgctctgcacgcacctatgtggaaactaaagcccagagagaaagtctgacttgccccacagccagtgagtgactgcagcagcaccagaatctggtctgtttcctgtttggctcttctaccactacggcttgggatctcgggcatggtggctttgccaatggtccttgttttgctgctggtcctgagcagaggtgagagtgaattggacgccaagatcccatccacaggggatgccacagaatggcggaatcctcacctgtccatgctggggtcctgccagccagccccctcctgccagaagtgcatcctctcacaccccagctgtgcatggtgcaagcaactgaacttcaccgcgtcgggagaggcggaggcgcggcgctgcgcccgacgagaggagctgctggctcgaggctgcccgctggaggagctggaggagccccgcggccagcaggaggtgctgcaggaccagccgctcagccagggcgcccgcggagagggtgccacccagctggcgccgcagcgggtccgggtcacgctgcggcctggggagccccagcagctccaggtccgcttccttcgtgctgagggatacccggtggacctgtactaccttatggacctgagctactccatgaaggacgacctggaacgcgtgcgccagctcgggcacgctctgctggtccggctgcaggaagtcacccattctgtgcgcattggttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcagaaaaaaaaaaaaaaaaaa

SEQ ID NO: 17 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 18 - DNA, 3879 bp, Homo sapiens
atcagacgcgcagaggaggcggggccgcggctggtttcctgccggggggcggctctgggccgccgagtcccctcctcccgcccctgaggaggaggagccgccgccacccgccgcgcccgacacccgggaggccccgccagcccgcgggagaggcccagcgggagtcgcggaacagcaggcccgagcccaccgcgccgggccccggacgccgcgcggaaaagatgaatttacaaccaattttctggattggactgatcagttcagtttgctgtgtgtttgctcaaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggcgtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcattccaattgtagctggtgtggttgctggaattgttcttattggccttgcattactgctgatatggaagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatgagtactgcccgtgcaaatcccacaacactgaatgcaaagtagcaatttccatagtcacagttaggtagctttagggcaatattgccatggttttactcatgtgcaggttttgaaaatgtacaatatgtataatttttaaaatgttttattattttgaaaataatgttgtaattcatgccagggactgacaaaagacttgagacaggatggttactcttgtcagctaaggtcacattgtgcctttttgaccttttcttcctggactattgaaatcaagcttattggattaagtgatatttctatagcgattgaaagggcaatagttaaagtaatgagcatgatgagagtttctgttaatcatgtattaaaactgatttttagctttacaaatatgtcagtttgcagttatgcagaatccaaagtaaatgtcctgctagctagttaaggattgttttaaatctgttattttgctatttgcctgttagacatgactgatgacatatctgaaagacaagtatgttgagagttgctggtgtaaaatacgtttgaaatagttgatctacaaaggccatgggaaaaattcagagagttaggaaggaaaaaccaatagctttaaaacctgtgtgccattttaagagttacttaatgtttggtaacttttatgccttcactttacaaattcaagccttagataaaagaaccgagcaattttctgctaaaaagtccttgatttagcactatttacatacaggccatactttacaaagtatttgctgaatggggaccttttgagttgaatttattttattatttttattttgtttaatgtctggtgctttctgtcacctcttctaatcttttaatgtatttgtttgcaattttggggtaagactttttttatgagtactttttctttgaagttttagcggtcaatttgcctttttaatgaacatgtgaagttatactgtggctatgcaacagctctcacctacgcgagtcttactttgagttagtgccataacagaccactgtatgtttacttctcaccatttgagttgcccatcttgtttcacactagtcacattcttgttttaagtgcctttagttttaacagttcactttttacagtgctatttactgaagttatttattaaatatgcctaaaatacttaaatcggatgtcttgactctgatgtattttatcaggttgtgtgcatgaaatttttatagattaaagaagttgaggaaaagcaaaaaaaaaa

SEQ ID NO: 19 - protein, 364 aa, Homo sapiens; identical to SEQ ID NO: 2

SEQ ID NO: 20 - protein, 778 aa, Homo sapiens
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 21 - protein, 412 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 22 - DNA, 2573 bp, Homo sapiens
acacacagccattgggggttgctcggatccgggactgccgcagggggtgccacagcagtgcctggcagcgtgggctgggaccttgtcactaaagcagagaagccacttcttctgggcccacgaggcagctgtcccatgctctgctgagcacggtggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccattccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaaggaagatggagctcccccatcccacacgcactgcactgccattgtcttttggttgccatggtcaccaaacaggaagtggacattctaagggaggagtactgaagagtgacggacttctgaggctgtttcctgctgctcctctgacttggggcagcttgggtcttcttgggcacctctctgggaaaacccagggtgaggttcagcctgtgagggctgggatgggtttcgtgggcccaagggcagacctttctttgggactgtgtggaccaaggagcttccatctagtgacaagtgacccccagctatcgcctcttgccttcccctgtggccactttccagggtggactctgtcttgttcactgcagtatcccaactgcaggtccagtgcaggcaataaatatgtgatggacaaacgatagcggaatccttcaaggtttcaaggctgtctccttcaggcagccttcccggaattctccatccctcagtgcaggatgggggctggtcctcagctgtctgccctcagcccctggccccccaggaagcctctttcatgggctgttaggttgacttcagttttgcctcttggacaacagggggtcttgtacatccttgggtgaccaggaaaagttcaggctatggggggccaaagggagggctgccccttccccaccagtgaccactttattccacttcctccattacccagttttggcccacagagtttggtcccccccaaacctcggaccaatatccctctaaacatcaatctatcctcctgttaaagaaaaaaaaaaa

SEQ ID NO: 23 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 24 - DNA, 2621 bp, Homo sapiens
ccttttttggcctcgacggcggcaacccagcctccctcctaacgccctccgcctttgggaccaaccaggggagctcaagttagtagcagccaaggagaggcgctgccttgccaagactaaaaagggaggggagaagagaggaaaaaagcaagaatcccccacccctctcccgggcggagggggcgggaagagcgcgtcctggccaagccgagtagtgtcttccactcggtgcgtctctctaggagccgcgcgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacacaaaaaa

SEQ ID NO: 25 - protein, 47 aa, Homo sapiens
MKFFMVLLPASLASTSLAILDVESGLLPQLSVLLSNRLRGKTCQTGP

SEQ ID NO: 26 - DNA, 297 bp, Homo sapiens
aactgatcctgctacagctccaccatgaagttcttcatggtcctgctgccagccagcctagcctccacctctctcgccatccttgatgttgaatctggcctccttccacaactctcagtactcctcagcaaccgcctcagagggaaaacttgccaaactggcccataaaatcgggaagaatctggacaaagcattgaaaggaatcataaactatctgaaaaacctaatccctagcgccaatgatgtcatgaggccctaatgatgagggtgcctgaattccaggctaggctgtgggag

SEQ ID NO: 27 - DNA, 6082 bp, Homo sapiens
ataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccggttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaattacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacacgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaa

(19) United States
(12) Patent Application Publication
(10) Pub. No.: US 2018/0142017 A1
(43) Pub. Date: May 24, 2018

(54) Methods for Treating Corneal and Conjunctival Inflammation and Inflammatory Disorders

(71) Applicants: Massachusetts Eye and Ear Infirmary, Boston, MA (US); The Children's Medical Center Corporation, Boston, MA (US)

(72) Inventors: Pedram Hamrah, Wellesley, MA (US); Ulrich von Andrian, Chestnut Hill, MA (US); Aslihan Turhan, West Roxbury, MA (US)

(21) Appl. No.: 15/669,673

(22) Filed: Aug. 4, 2017

Related U.S. Application Data
(63) Continuation of application No. 14/379,397, filed on Aug. 18, 2014, now abandoned.
(60) Provisional application No. 61/601,300, filed on Feb. 21, 2012.

(51) Int. Cl. C07K 16/28; A61K 45/06; A61K 39/395

(52) U.S. Cl. CPC C07K 16/2803; A61K 45/06; C07K 2317/54; C07K 2317/55; A61K 39/3955; A61K 2039/505; C07K 16/2839; C07K 2317/21; C07K 2317/24; C07K 2317/76

(57) ABSTRACT

Provided herein are methods for reducing corneal inflammation, reducing inflammatory cell (e.g., dendritic cell) recruitment to the cornea, and treating an corneal inflammatory disorder in a subject that include administering to the subject one or more of a MadCAM-1 antagonist, an α4β7 integrin antagonist, a L-selectin antagonist, and an E-selectin antagonist. Also provided are compositions containing one or more of a MadCAM-1 antagonist, an α4β7 integrin antagonist, a L-selectin antagonist, and an E-selectin antagonist, and kits containing these compositions.

### CLAIM OF PRIORITY

This application is the continuation of U.S. application Ser. No. 14/379,397, filed Aug. 18, 2014, which is a U.S. national stage under 35 U.S.C. § 371 of International Patent Application Number PCT/US2013/027172, filed on Feb. 21, 2013, which claims priority to U.S. Provisional Patent Application Ser. No. 61/601,300, filed on Feb. 21, 2012. The entire contents of the foregoing are hereby incorporated by reference.

### FEDERALLY SPONSORED RESEARCH

This invention was made with Government support under grant number NIH K08-EY020575 awarded by the National Institutes of Health. The Government has certain rights in the invention.

### BACKGROUND OF THE INVENTION

Corneal inflammation can be caused by a variety of factors including, but not limited to, bacterial infection, fungal infection, parasite infection, virus infection (e.g., herpes simplex and herpes zoster), allergies, dry eye disorder, Fuchs' dystrophy, keratoconus, amyloidosis, lattice dystrophy, Stevens Johnson syndrome, physical corneal injury, Behcet's disease, and contact lens wear. Corneal inflammatory disorders, such as keratitis (e.g., infectious and non-infectious keratitis), are characterized by an elevated level of corneal inflammation. The symptoms of corneal inflammation are thought to be mediated or triggered by the recruitment of various immunological cells (e.g., dendritic cells) to the cornea.

Adhesion molecules play a role in the recruitment of immunological cells to different tissues in the body. Adhesion molecules can be categorized according to their structure and function. Four major families are distinguished: the selectins, the sialomucins, the integrins, and the immunoglobulin superfamily. Little is known regarding the adhesion molecules that play a role in the recruitment of dendritic cells to the cornea.

### SUMMARY OF THE INVENTION

The invention is based, in part, on the discovery that anti-MadCAM-1, anti-E-selectin, anti-L-selectin, and anti-α4β7 integrin antibodies decrease the migration of dendritic cells to the cornea in a mouse model of corneal inflammation, and decrease the sticking and rolling of dendritic cells in the limbal vessel (a vessel that is, e.g., proximal to both the cornea and the conjunctiva). In view of this discovery, provided herein are methods for reducing corneal and/or conjunctival inflammation, reducing inflammatory cell (e.g., dendritic cell) recruitment to the cornea and/or conjunctiva, and treating a corneal and/or conjunctival inflammatory disorder in a subject that include administering to the subject one or more (e.g., two, three, four, or five) of a MadCAM-1 antagonist, an α4β7 integrin antagonist, a L-selectin antagonist, and an E-selectin antagonist. Also provided are compositions that contain one or more (e.g., two, three, four, or five) of a MadCAM-1 antagonist, an α4β7 integrin antagonist, a L-selectin antagonist, and an E-selectin antagonist, and kits that contain these compositions.

Provided herein are methods of reducing corneal inflammation in a subject that include administering to a subject having corneal inflammation one or more (e.g., two, three, four, or five) of a MadCAM-1 antagonist, an α4β7 integrin antagonist, a L-selectin antagonist, and an E-selectin antagonist in an amount sufficient to reduce corneal inflammation in the subject. In some embodiments, the corneal inflammation is chronic corneal inflammation. In some embodiments, the corneal inflammation is acute corneal inflammation. In some embodiments, the corneal inflammation is caused by bacterial infection, fungal infection, parasite infection, viral infection, allergies, dry eye disorder, Fuchs' dystrophy, keratoconus, amyloidosis, lattice dystrophy, Stevens Johnson syndrome, physical corneal injury, Behcet's disease, contact lens wear, corneal graft rejection, dry eye syndrome, or immune keratitis (e.g., peripheral ulcerative keratitis). Some embodiments further include administering to the subject one or more anti-inflammatory agents. Some embodiments further include selecting a subject having eye inflammation or a corneal inflammatory disorder. In some embodiments of any of the methods described herein, the subject does not have ulcerative colitis, multiple sclerosis, and/or Crohn's disease.

Also provided are methods of reducing dendritic cell recruitment to the cornea that include administering to a subject having corneal inflammation one or more (e.g., two, three, four, or five) of a MadCAM-1 antagonist, an α4β7 integrin antagonist, a L-selectin antagonist, and an E-selectin antagonist in an amount sufficient to reduce dendritic cell recruitment to the cornea. In some embodiments, the dendritic cell recruitment is recruitment of dendritic cells to the corneal epithelium. In some embodiments, the dendritic cell recruitment is recruitment of dendritic cells to the anterior or posterior stroma of the cornea. Some embodiments further include selecting a subject having eye inflammation or a corneal inflammatory disorder. Some embodiments further include administering to the subject one or more anti-inflammatory agents.

Also provided are methods of treating a corneal inflammatory disorder in a subject that include administering to a subject having a corneal inflammatory disorder one or more (e.g., two, three, four, or five) of a MadCAM-1 antagonist, an α4β7 integrin antagonist, a L-selectin antagonist, and an E-selectin antagonist in an amount sufficient to decrease corneal inflammation. In some embodiments, the corneal inflammatory disorder is keratitis. In some embodiments, the keratitis is non-infectious keratitis. In some embodiments, the keratitis is infectious keratitis. Some embodiments further include selecting a subject having a corneal inflammatory disorder. Some embodiments further include administering to the subject one or more anti-inflammatory agents.

In some embodiments of the methods described herein, the MadCAM-1 antagonist is an antibody or an antigen-binding antibody fragment that binds specifically to MadCAM-1. In some embodiments, the antibody is a fully human antibody or humanized antibody. In some embodiments, the antigen-binding antibody fragment is selected from the group of: a Fab fragment, a F(ab′)2 fragment, and a scFv fragment.

In some embodiments of the methods described herein, the α4β7 integrin antagonist is an antibody or an antigen-binding antibody fragment that binds specifically to α4β7 integrin. In some embodiments, the antibody is a fully human antibody or a humanized antibody. In some embodiments, the antigen-binding antibody fragment is selected from the group of: a Fab fragment, a F(ab′)2 fragment, and a scFv fragment.

In some embodiments of the methods described herein, the α4β7 integrin antagonist is a small molecule.

In some embodiments of the methods described herein, the L-selectin antagonist is an antibody or an antigen-binding antibody fragment that binds specifically to L-selectin. In some embodiments, the antibody is a fully human antibody or a humanized antibody. In some embodiments, the antigen-binding antibody fragment is selected from the group of: a Fab fragment, a F(ab′)2 fragment, and a scFv fragment.

In some embodiments of the methods described herein, the E-selectin antagonist is an antibody or an antigen-binding antibody fragment that binds specifically to E-selectin. In some embodiments, the antibody is a fully human antibody or a humanized antibody. In some embodiments, the antigen-binding antibody fragment is selected from the group of: a Fab fragment, a F(ab′)2 fragment, and a scFv fragment.

In some embodiments of the methods described herein, the administering is ocular administration.

In some embodiments of the methods described herein, one or more doses of the one or more of a MadCAM-1 antagonist, an α4β7 integrin antagonist, a L-selectin antagonist, and an E-selectin antagonist are administered to the subject. In some embodiments, a dose of the one or more of a MadCAM-1 antagonist, an α4β7 integrin antagonist, a L-selectin antagonist, and an E-selectin antagonist is administered to the subject at least once a month (e.g., at least once every two weeks or at least once a day).

In some embodiments of the methods described herein, at least one MadCAM-1 antagonist and at least one α4β7 integrin antagonist are administered to the subject. In some embodiments, the at least one MadCAM-1 antagonist and the at least one α4β7 integrin antagonist are present in the same formulation.

Also provided herein are methods of using one or more of a MadCAM-1 antagonist, an α4β7 integrin antagonist, a L-selectin antagonist, and an E-selectin antagonist for reducing corneal inflammation in a subject. Also provided herein are one or more of a MadCAM-1 antagonist, an α4β7 integrin antagonist, a L-selectin antagonist, and an E-selectin antagonist for use in reducing corneal inflammation in a subject, and/or for use in the manufacture of a medicament for reducing corneal inflammation in a subject.

Also provided herein are methods of using one of more of a MadCAM-1 antagonist, an α4β7 integrin antagonist, a L-selectin antagonist, and an E-selectin antagonist for reducing dendritic cell recruitment to the cornea in a subject. Also provided herein are one or more of a MadCAM-1 antagonist, an α4β7 integrin antagonist, a L-selectin antagonist, and an E-selectin antagonist for use in reducing dendritic cell recruitment to the cornea in a subject, and/or for use in the manufacture of a medicament for reducing dendritic cell recruitment to the cornea in a subject.

Also provided herein are methods of using one or more of a MadCAM-1 antagonist, an α4β7 integrin antagonist, a L-selectin antagonist, and an E-selectin antagonist for treating a corneal inflammatory disorder in a subject. Also provided herein are one or more of MadCAM-1 antagonist, an α4β7 integrin antagonist, a L-selectin antagonist, and an E-selectin antagonist for use in treating a corneal inflammatory disorder in a subject, and/or for use in the manufacture of a medicament for treating a corneal inflammatory disorder in a subject.

Also provided are compositions containing at least one MadCAM-1 antagonist and at least one α4β7 integrin antagonist, where the at least one MadCAM-1 antagonist and the at least one α4β7 integrin antagonist are present in amounts that together are sufficient to reduce corneal inflammation in a subject. In some embodiments, the at least one MadCAM-1 antagonist or the at least one α4β7 integrin antagonist is an antibody or an antigen-binding antibody fragment. In some embodiments, the antibody is a fully human antibody or a humanized antibody. In some embodiments, the antigen-binding antibody fragment is selected from the group of: a Fab fragment, a F(ab′)₂ fragment, and a scFv fragment. In some embodiments, the at least one α4β7 integrin antagonist is a small molecule.

Also provided are kits containing any of the compositions described herein, for use in a method described herein, and optionally instructions for administering the composition to a subject having corneal inflammation or a corneal inflammatory disorder.

Also provided are kits for use in a method described herein, wherein the kits contain a composition comprising at least one MadCAM-1 antagonist and at least one α4β7 integrin antagonist; and optionally instructions for use of the composition in any of the methods described herein, where the at least one MadCAM-1 antagonist and the at least one α4β7 integrin antagonist are present in the composition in amounts that together are sufficient to reduce corneal inflammation in a subject.

As used herein, the term “corneal inflammation” is generally meant the presence or observation of two or more (e.g., three, four, or five) of the following in a subject: an elevated number of T-lymphocytes (e.g., effector T-cells) in a cornea, an elevated number of dendritic cells in a cornea, an elevated number of macrophages in a cornea, an elevated number of eosinophils in a cornea, an elevated number of mast cells in a cornea, an elevated number of B-cells in a cornea, an elevated number of stimulated monocytes in a cornea, an elevated number of natural killer cells in a cornea, an elevated level of redness in a cornea, pain in an eye, irritation, itchiness, burning, and/or dryness of a cornea, excess tears or other discharge from an eye, difficulty opening an eyelid, blurred vision, sensitivity to light, and swelling around the eye (e.g., as compared to the levels in the same subject prior to corneal inflammation, a subject not having an eye disorder (a healthy subject), or a threshold value). Corneal inflammation can be caused by a variety of different factors. Non-limiting examples of such causative factors are described herein. Additional causative factors are known in the art.

By the term “corneal inflammatory disorder” is meant a disorder of the eye that is characterized by two of more of the following features: an elevated number of T-lymphocytes (e.g., effector T-cells) in a cornea, an elevated number of dendritic cells in a cornea, an elevated number of macrophages in a cornea, an elevated number of stimulated monocytes in a cornea, an elevated number of eosinophils in a cornea, an elevated number of mast cells in a cornea, an elevated number of B-cells in a cornea, an elevated number of natural killer cells in a cornea, an elevated level of redness in a cornea, pain in an eye, irritation, itchiness, burning, and/or dryness of a cornea, excess tears or other discharge from an eye, difficulty opening an eyelid, blurred vision, sensitivity to light, and swelling around the eye (e.g., as compared to the levels in the same subject prior to corneal inflammation, a subject not having an eye disorder (a healthy subject), or a threshold value). A non-limiting example of a corneal inflammatory disorder is keratitis (e.g., infectious keratitis or non-infectious keratitis). Additional examples of corneal inflammatory disorders are described herein and are known in the art. Methods for identifying or diagnosing a corneal inflammatory disorder in a subject are described herein and are known in the art.

As used herein, the term “conjunctival inflammation” is generally meant the presence or observation of two or more (e.g., three, four, or five) of the following in a subject: an elevated number of T-lymphocytes (e.g., effector T-cells) in a conjunctiva, an elevated number of dendritic cells in a conjunctiva, an elevated number of macrophages in a conjunctiva, an elevated number of stimulated monocytes in a conjunctiva, an elevated number of natural killer cells in a conjunctiva, an elevated number of B-cells in a conjunctiva, an elevated number of eosinophils in a conjunctiva, an elevated number of mast cells in a conjunctiva, an elevated level of redness in a white of an eye or inner eyelid, pain in an eye, irritation, itchiness, burning, and/or dryness of an eye, excess tears or other discharge from an eye, difficulty opening an eyelid, blurred vision, sensitivity to light, and swelling around an eye (e.g., as compared to the levels in the same subject prior to conjunctival inflammation, a subject not having an eye disorder (a healthy subject), or a threshold value). Conjunctival inflammation can be caused by a variety of different factors (e.g, viruses, bacteria (e.g., gonorrhea or chlamydia), irritants (e.g., shampoos, dirt, smoke, or chlorine), an allergen, or contact lens wear. Additional causative factors are known in the art. A subject can be diagnosed as having conjunctival inflammation by observing or detecting one or more of the symptoms described herein.

By the term “conjunctival inflammatory disorder” is meant is meant a disorder of the eye that is characterized by two of more of the following features: an elevated number of T-lymphocytes (e.g., effector T-cells) in a conjunctiva, an elevated number of dendritic cells in a conjunctiva, an elevated number of macrophages in a conjunctiva, an elevated number of stimulated monocytes in a conjunctiva, an elevated number of B-cells in a conjunctiva, an elevated number of natural killer cells in a conjunctiva, an elevated number of eosinophils in a conjunctiva, an elevated number of mast cells in a conjunctiva, an elevated level of redness in a white of an eye or inner eyelid, pain in an eye, irritation, itchiness, burning, and/or dryness of an eye, excess tears or other discharge from an eye, difficulty opening an eyelid, blurred vision, sensitivity to light, and swelling around an eye (e.g., as compared to the levels in the same subject prior to conjunctival inflammation, a subject not having an eye disorder (a healthy subject), or a threshold value). Non-limiting example of a conjunctival inflammatory disorders are viral conjunctivitis, bacterial conjunctivis, fungal conjunctivitis, parasitic conjunctivitis, or allergic conjunctivitis. Additional examples of conjunctival inflammatory disorders are known in the art. Methods for identifying or diagnosing a conjunctival inflammatory disorder are known in the art.

By the term “inflammatory cell” is meant a cell that contributes to one or more of the symptoms of a corneal inflammatory disorder or a conjunctival inflammatory disorder described herein. In some embodiments, the inflammatory cell expresses α4β7 in its plasma membrane. Non-limiting examples of inflammatory cells include dendritic cells, effector T-cells, eosinophils, B-cells, natural killer cells, mast cells, stimulated monocytes, macrophages, eosinophils, and mast cells.

By the term “MadCAM-1” or “mucosal vascular addressin cell adhesion molecule 1” is meant a polypeptide that contains a contiguous sequence (e.g., at least 7, 15, 20, 30, 40, 50, 60, 70, 80, 90, or 100 amino acids) that is at least 95% (e.g., at least 96%, 97%, 98%, 99%, or 100%) identical to a wild type form of MadCAM-1 (e.g., precursor or processed MadCAM-1, e.g., any one of SEQ ID NOS: 1-7 and 11).

By the term “MadCAM-1 antagonist” is meant an agent that specifically binds to a polypeptide that has a sequence at least 95% identical to a wild type form of MadCAM-1 (e.g., precursor or processed MadCAM-1, e.g., any one of SEQ ID NOS: 1-7 and 11) that has the ability to decrease the binding of MadCAM-1 to one of its natural cognate receptors (e.g., α4β7 integrin and L-selectin). Non-limiting examples of MadCAM-1 antagonists are antibodies or an antigen-binding antibody fragments that specifically bind to MadCAM-1, or soluble L-selectin molecules, soluble α4β1 integrin agents, or soluble α4β7 integrin agents.

By the term “soluble MadCAM-1 molecule” is meant a molecule that contains an amino acid sequence that is at least 95% identical to a contiguous sequence in a wild type form of MadCAM-1 (e.g., a precursor or processed MadCAM-1, e.g., any one of SEQ ID NOS: 1-7 and 11) that is soluble at a physiological pH and has the ability to specifically bind to α4β7, L-selectin, and/or α4β1. In some embodiments, the soluble MadCAM-1 molecule lacks its signal sequence, its transmembrane domain, and its cytoplasmic domain. In some embodiments, the soluble MadCAM-1 molecule contains an additional amino acid sequence (e.g., a sequence that stabilizes the protein or increases the protein's half-life in vivo, e.g., an Fc region or bovine serum albumin).

By the term “soluble L-selectin molecule” is meant a molecule that contains an amino acid sequence that is at least 95% identical to a contiguous sequence in a wild type form of L-selectin (e.g., precursor or processed L-selectin, e.g., any one of SEQ ID NOS: 10 and 19) that is soluble at a physiological pH and has the ability to specifically bind to MadCAM-1, CD34, PSGL-1, and/or GlyCAM-1. In some embodiments, the soluble L-selectin molecule lacks its signal sequence, its transmembrane domain, and its cytoplasmic domain. In some embodiments, the soluble L-selectin molecule contains an additional amino acid sequence (e.g., a sequence that stabilizes the protein or increases the protein's half-life in vivo, e.g., an Fc region or bovine serum albumin).

By the term “soluble α4β7 agent” is meant a composition that contains a protein (e.g., a single polypeptide or a heterodimeric protein) that contains an amino acid sequence that is at least 95% identical to a contiguous sequence in a wild type form of α4 integrin (e.g., precursor or processed form of α4, e.g., any one of SEQ ID NOS: 8 and 12) and an amino acid sequence that is at least 95% identical to a contiguous sequence in a wild type form of β7 integrin (e.g., precursor or processed form of β7, e.g., any one of SEQ ID NOS: 9 and 13), that is soluble at physiological pH and has the ability to specifically bind to MadCAM-1. In some embodiments, the amino acid sequence that is at least 95% identical to a contiguous sequence in α4 integrin lacks the signal sequence, the transmembrane domain, and cytoplasmic domain of α4 integrin, and/or the amino acid sequence that is at least 95% identical to a contiguous sequence in β7 integrin lacks the signal sequence, the transmembrane domain, and cytoplasmic domain of β7 integrin. Non-limiting examples of soluble α4β7 agents are described herein. In some embodiments, the soluble α4β7 agent contains a polypeptide that contains an additional amino acid sequence (e.g., a sequence that stabilizes the polypeptide or increases the polypeptide's half-life in vivo, e.g., an Fc region or bovine serum albumin).

By the term “soluble α4β1 agent” is meant a protein (e.g., a single polypeptide or a heterodimeric protein) that contains an amino acid sequence that is at least 95% identical to a contiguous sequence in a wild type form of α4 integrin (e.g., precursor or processed α4 integrin, e.g., any one of SEQ ID NOS: 8 and 12) and an amino acid sequence that is at least 95% identical to a contiguous sequence in a wild type form of β1 integrin (e.g., precursor or processed β1 integrin, e.g., any one of SEQ ID NOS: 17 and 20), that is soluble at physiological pH and has the ability to specifically bind to MadCAM-1. In some embodiments, the amino acid sequence that is at least 95% identical to a contiguous sequence in α4 integrin lacks the signal sequence, the transmembrane domain, and cytoplasmic domain of α4 integrin, and/or the amino acid sequence that is at least 95% identical to a contiguous sequence in β1 integrin lacks the signal sequence, the transmembrane domain, and cytoplasmic domain of β1 integrin. Non-limiting examples of soluble α4β1 agents are described herein. In some embodiments, the soluble α4β1 agent contains a polypeptide that contains an additional amino acid sequence (e.g., a sequence that stabilizes the polypeptide or increases the polypeptide's half-life in vivo, e.g., an Fc region or bovine serum albumin).

By the term “α4β7 integrin” is meant a heterodimeric protein made of a protein having a sequence that is at least 95% identical to a wild type form of α4 integrin (e.g., precursor or processed α4 integrin, e.g., any one of SEQ ID NOS: 8 and 12) and a protein having a sequence that is at least 95% identical to a wild type form of β7 integrin (e.g., precursor or processed β7 integrin, e.g., any one of SEQ ID NOS: 9 and 13).

By the term “α4β7 integrin antagonist” is meant a molecule that specifically binds to the heterodimeric protein that contains a polypeptide having a sequence at least 95% (e.g., at least 96%, 97%, 98%, 99%, or 100%) identical to a wild type form of α4 integrin (e.g., precursor or processed α4 integrin, e.g., any one of SEQ ID NOS: 8 and 12) and a polypeptide having a sequence at least 95% (e.g., 96%, 97%, 97%, 99%, or 100%) identical to a wild type form of β7 integrin (e.g., precursor or processed β7 integrin, e.g., any one of SEQ ID NOS: 9 and 13) that has the ability to decrease (e.g., a significant or observable decrease) the binding of α4β7 integrin to one of its natural cognate receptors (e.g., MadCAM-1). Non-limiting examples of α4β7 integrin antagonists are antibodies or an antigen-binding antibody fragments that specifically bind to α4β7, α4 integrin, β7 integrin, or soluble MadCAM-1 molecules. Additional examples of α4β7 integrin antagonists are small molecules (e.g., the small molecule α4β7 integrin antagonists described herein or known in the art).

By the term “L-selectin” is meant a polypeptide that contains a contiguous sequence (e.g., at least 7, 15, 20, 30, 40, 50, 60, 70, 80, 90, or 100 amino acids) that is at least 95% (e.g., at least 96%, 97%, 98%, 99%, or 100%) identical to a wild type form of L-selectin (e.g., precursor or processed L-selectin, e.g., any one of SEQ ID NOS: 10 and 19).

By the term “L-selectin antagonist” is meant is meant an agent that specifically binds to a protein having a sequence at least 95% (e.g., at least 96%, 97%, 98%, 99%, or 100%) identical to a wild type form of L-selectin integrin (e.g., precursor or processed L-selectin, e.g., any one of SEQ ID NOS: 10 and 19) that has the ability to decrease (e.g., a significant or observable decrease) the binding of L-selectin to one of its natural cognate receptors (e.g., MadCAM-1, CD34, PSGL-1, and/or GlyCAM-1). Non-limiting examples of L-selectin antagonists are antibodies or an antigen-binding antibody fragments that specifically bind to L-selectin, or a soluble MadCAM-1 agent, a soluble CD34 molecule, a soluble PSGL-1 molecule, or a soluble GlyCAM-1 agent. Additional examples of L-selectin antagonists are small molecules (e.g., the small molecule L-selectin antagonists described herein or known in the art).

By the term “soluble PSGL-1 molecule” is meant a molecule that contains an amino acid sequence that is at least 95% (e.g., at least 96%, 97%, 98%, 99%, or 100%) identical to a contiguous sequence in a wild type form of PSGL-1 (e.g., precursor or processed PSGL-1, e.g., SEQ ID NO: 21) that is soluble at a physiological pH and has the ability to specifically bind to L-selectin. In some embodiments, the soluble PSGL-1 molecule lacks its signal sequence, its transmembrane domain, and its cytoplasmic domain. In some embodiments, the soluble PSGL-1 molecule contains an additional amino acid sequence (e.g., a sequence that stabilizes the protein or increases the protein's half-life in vivo, e.g., an Fc region or bovine serum albumin).

By the term “soluble CD34 molecule” is meant a molecule that contains an amino acid sequence that is at least 95% (e.g., at least 96%, 97%, 98%, 99%, or 100%) identical to a contiguous sequence in a wild type form of CD34 (e.g., precursor or processed CD34, e.g., SEQ ID NO: 23) that is soluble at a physiological pH and has the ability to specifically bind to L-selectin. In some embodiments, the soluble CD34 molecule lacks its signal sequence, its transmembrane domain, and its cytoplasmic domain. In some embodiments, the soluble CD34 molecule contains an additional amino acid sequence (e.g., a sequence that stabilizes the protein or increases the protein's half-life in vivo, e.g., an Fc region or bovine serum albumin).

By the term “soluble GlyCAM-1 molecule” is meant a molecule that contains an amino acid sequence that is at least 95% (e.g., 96%, 97%, 98%, 99%, or 100%) identical to a contiguous sequence in a wild type form of GlyCAM-1 (e.g., precursor or processed GlyCAM-1, e.g., SEQ ID NO: 25) that is soluble at a physiological pH and has the ability to specifically bind to L-selectin. In some embodiments, the soluble GlyCAM-1 molecule lacks its signal sequence. In some embodiments, the soluble GlyCAM-1 molecule contains an additional amino acid sequence (e.g., a sequence that stabilizes the protein or increases the protein's half-life in vivo, e.g., an Fc region or bovine serum albumin).

By the term “chronic corneal inflammation” is meant the observance or a detectable level of at least two or more (e.g., at least three or four) of an elevated number of T-lymphocytes (e.g., effector T-cells) in a cornea, an elevated number of dendritic cells in a cornea, an elevated number of macrophages in a cornea, an elevated number of stimulated monocytes in a cornea, an elevated level of natural killer cells in a cornea, an elevated level of B-cells in a cornea, an elevated number of eosinophils in a cornea, an elevated number of mast cells in a cornea, an elevated level of redness in a cornea, pain in an eye, irritation, itchiness, burning, and/or dryness of a cornea, excess tears or other discharge from an eye, difficulty opening an eyelid, blurred vision, sensitivity to light, and swelling around the eye (e.g., as compared to the levels in the same subject prior to corneal inflammation, a subject not having an eye disorder (a healthy subject), or a threshold value) in the subject for at least two weeks.

By the term “acute corneal inflammation” is meant the observance or a detectable level of at least two or more (e.g., at least three or four) of an elevated number of T-lymphocytes (e.g., effector T-cells) in a cornea, an elevated number of dendritic cells in a cornea, an elevated number of macrophages in a cornea, an elevated number of stimulated monocytes in a cornea, an elevated level of B-cells in a cornea, an elevated level of natural killer cells in a cornea, an elevated number of eosinophils in a cornea, an elevated number of mast cells in a cornea, an elevated level of redness in a cornea, pain in an eye, irritation, itchiness, burning, and/or dryness of a cornea, excess tears or other discharge from an eye, difficulty opening an eyelid, blurred vision, sensitivity to light, and swelling around the eye (e.g., as compared to the levels in the same subject prior to corneal inflammation, a subject not having an eye disorder (a healthy subject), or a threshold value) in the subject for two weeks or less.

By the term “chronic conjunctival inflammation” is meant the observance or a detectable level of at least two or more (e.g., at least three or four) of an elevated number of T-lymphocytes (e.g., effector T-cells) in a conjunctiva, an elevated number of dendritic cells in a conjunctiva, an elevated number of macrophages in a conjunctiva, an elevated number of stimulated monocytes in a conjunctiva, an elevated level of natural killer cells in a conjunctiva, an elevated level of B-cells in a conjunctiva, an elevated number of eosinophils in a conjunctiva, an elevated number of mast cells in a conjunctiva, an elevated level of redness in the white of an eye or an eyelid, pain in an eye, irritation, itchiness, burning, and/or dryness of an eye, excess tears or other discharge from an eye, difficulty opening an eyelid, blurred vision, sensitivity to light, and swelling around the eye (e.g., as compared to the levels in the same subject prior to conjunctival inflammation, a subject not having an eye disorder (a healthy subject), or a threshold value) in the subject for at least two weeks.

By the term “acute conjunctival inflammation” is meant the observance or a detectable level of at least two or more (e.g., at least three or four) of an elevated number of T-lymphocytes (e.g., effector T-cells) in a conjunctiva, an elevated number of dendritic cells in a conjunctiva, an elevated number of macrophages in a conjunctiva, an elevated number of stimulated monocytes in a conjunctiva, an elevated level of natural killer cells in a conjunctiva, an elevated level of B-cells in a conjunctiva, an elevated number of eosinophils in a conjunctiva, an elevated number of mast cells in a conjunctiva, an elevated level of redness in the white of an eye or an eyelid, pain in an eye, irritation, itchiness, burning, and/or dryness of an eye, excess tears or other discharge from an eye, difficulty opening an eyelid, blurred vision, sensitivity to light, and swelling around the eye (e.g., as compared to the levels in the same subject prior to conjunctival inflammation, a subject not having an eye disorder (a healthy subject), or a threshold value) in the subject for two weeks or less.

By the term “dendritic cell” is meant a bone-marrow derived corpuscular cell with tree-like processes that can, e.g., act as antigen-presenting cells (e.g., they can phagocytose or endocytose an antigen, and transport and present the antigen to T-lymphocyte(s)). The normal (healthy) cornea (e.g., central cornea) contains immature/precursor-type dendritic cells under steady state conditions; however, these cells can upregulate maturation markers, such as MHC class II molecules, and can increase in reflectivity and size.

By the phrase “inflammatory cell recruitment to the cornea” is meant the migration of an inflammatory cell (e.g., any of the inflammatory cells described herein) from a blood vessel (e.g., limbal vessel) into the cornea. In some embodiments, the inflammatory cell migrates to the corneal epithelium. In some embodiments, the inflammatory cell migrates to the anterior stroma or posterior stroma of the cornea.

By the phrase, “dendritic cell recruitment to the cornea” is meant the migration of a dendritic cell from a blood vessel (e.g., limbal vessel) into the cornea. In some embodiments, the dendritic cell migrates to the corneal epithelium. In some embodiments, the dendritic cell migrates to the anterior stroma or posterior stroma of the cornea.

By the phrase “inflammatory cell recruitment to the conjunctiva” is meant the migration of an inflammatory cell (e.g., any of the inflammatory cells described herein) from a blood vessel (e.g., limbal vessel) into the conjunctiva.

By the phrase, “dendritic cell recruitment to the conjunctiva” is meant the migration of a dendritic cell from a blood vessel (e.g., limbal vessel) into the conjunctiva.

As used herein, the term “antibody” means a protein that generally contains heavy chain polypeptides and light chain polypeptides. Antigen recognition and binding occurs within the variable regions of the heavy and light chains. Single domain antibodies having one heavy chain and one light chain, and heavy chain antibodies devoid of light chains, are also known. A given antibody comprises one of five different types of heavy chains, called alpha, delta, epsilon, gamma, and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3, and IgG4) and IgM, respectively. A given antibody also comprises one of two types of light chains, called kappa or lambda, the categorization of which is based on the amino acid sequence of the light chain constant domains. IgG IgD, and IgE antibodies generally contain two identical heavy chains and two identical light chains and two antigen combining domains, each composed of a heavy chain variable region (VH) and a light chain variable region (VL). Generally IgA antibodies are composed of two monomers, each monomer composed of two heavy chains and two light chains (as for IgG, IgD, and IgE antibodies). In this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). In this way the secreted IgM molecule has ten antigen-binding domains, each again composed of a VH and a VL. A cell surface form of IgM also exists and this has a two heavy chain/two light chain structure similar to IgG IgD, and IgE antibodies.

As used herein, the term “chimeric antibody” refers to an antibody that has been engineered to comprise at least one human constant region. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region. Chimeric antibodies are typically less immunogenic to humans, relative to non-chimeric antibodies, and thus offer therapeutic benefits in certain situations. Those skilled in the art will be aware of chimeric antibodies, and will also be aware of suitable techniques for their generation. See, for example, U.S. Pat. Nos. 4,816,567; 4,978,775; 4,975,369; and U.S. Pat. No. 4,816,397.

As used herein, the term “fully human antibodies” are antibodies or antigen binding fragments of antibodies that contain only human-derived amino acid sequences. For example, a fully human antibody may be produced from a human B-cell or a human hybridoma cell. In additional embodiments, the antibody may be produced from a transgenic animal that contains the locus for a human heavy chain immunoglobulin and a human light chain immunoglobulin, or contains a nucleic acid that encodes the heavy and light chains of a specific human antibody.

“Antigen-binding antibody fragment” or “antibody fragment” as the terms are used herein refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments can include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

“Humanized antibody” as the term is used herein refers to an antibody that has been engineered to comprise one or more human framework regions in the variable region together with non-human (e.g., mouse, rat, or hamster) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations. Humanized antibodies are known in the art, and suitable techniques for generating humanized antibodies are also known. See for example, Hwang et al., Methods 36:35, 2005; Queen et al., Proc. Natl. Acad. Sci. U.S.A. 86:10029-10033, 1989; Jones et al., Nature 321:522-25, 1986; Riechmann et al., Nature 332:323-27, 1988; Verhoeyen et al., Science 239:1534-36, 1988; Orlandi et al., Proc. Natl. Acad. Sci. U.S.A. 86:3833-3837, 1989; U.S. Pat. Nos. 5,225,539; 5,530,101; 5,585,089; 5,693,761; 5,693,762; and 6,180,370; and WO 90/07861.

By the term “anti-inflammatory agent” is meant an agent that is administered to a subject in order to reduce one or more symptoms of including: pain, heat, redness, swelling, in a tissue in a subject, bradykinin levels, lysosome enzyme levels, histamine levels, interferon-γ levels, IL-8 levels, leukotriene B4 levels, prostaglandin levels, TNF-α levels, and IL-1 levels, reduce the migration of inflammatory cells (e.g., any of the inflammatory cells described herein) into a tissue (e.g., a cornea or conjunctiva), and reduce the number of inflammatory cells (e.g., any of the inflammatory cells described herein) present in a tissue (e.g., a cornea or conjunctiva). Non-limiting examples of anti-inflammatory agents include non-steroidal anti-inflammatory agents (NSAIDS) (e.g., aspirin, diflusinal, salsalate, ibuprofen, naproxen, fenoprofen, ketoprofen, dexketoprofen, flurbiprofen, oxaprozin, loxoprofen, indomethacin, sulindac, etodolac, ketorolac, diclofenac, nabumetone, piroxicam, meloxicam, tenoxicam, droxicam, lornoxicam, isoxicam, mefanamic acid, meclofenamic acid, flufenamic acid, tolfenamic acid, celecoxib, rofecoxib, valdecoxib, parecoxib, lumiracoxib, etoricoxib, firocoxib, nimesulide, and licofelone), steroids (e.g., hydrocortisone, cortisone, prednisone, prednisolone, methylprednisolone, dexamethasone, betamethasone, triamcinolone, beclometasone, fludrocortisone, deoxycorticosterone, and aldosterone) and calcineurin inhibitors (e.g., cyclosporin, tacrolimus, and sirolimus).

By the term “subject” is meant any mammal (e.g., a human, mice, rat, and rabbit). Other definitions appear in context throughout this disclosure. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a diagram of the eye showing the inflammatory cell populations present in the corneal epithelium and the corneal stroma under normal conditions (normal cornea) and during inflammation (inflamed cornea).

FIG. 2 is a diagram showing the injection of calcein-labeled dendritic cells into the carotid artery of a mouse and the intravital imaging of the cornea of the mouse. The mice used to perform these experiments can be control mice in which inflammation has not been induced or a mouse eye inflammation model.

FIG. 3 is a picture of an exemplary suture made in a mouse eye inflammation model.

FIG. 4A is an in vitro confocal micrograph showing the limbal vessel in a control (untreated) mouse following injection of FITC-dextran.

FIG. 4B is an in vitro confocal micrograph showing the presence of calcein-labeled dendritic cells present in the limbal vessel of a mouse following induction of inflammation (suture-induced inflammation).

FIG. 4C is an in vitro confocal micrograph showing the presence of calcein-labeled dendritic cells present in the limbal vessel of a mouse that received an anti-VCAM-1 antibody prior to induction of eye inflammation (suture-induced inflammation).

FIG. 5 is a graph showing the percentage of rolling calcein-labeled dendritic cells in the limbal vessel compared to the total number of passing calcein-labeled dendritic cells in the limbal vessel in a mouse receiving no treatment or receiving an anti-P-selection antibody (anti-P-Sel), an anti-L-selectin antibody (anti-L-Sel), an anti-E-selectin antibody (anti-E-Sel), or an anti-PSGL-1 antibody (anti-PSGL-1).

FIG. 6 is a graph showing the percentage of rolling calcein-labeled dendritic cells in the limbal vessel compared to the total number of passing calcein-labeled dendritic cells in the limbal vessel in a mouse eye inflammation model receiving no treatment or receiving an anti-P-selectin antibody (anti-P-Sel), an anti-L-selectin antibody (anti-L-Sel), a combination of an anti-P-selectin antibody and an anti-L-selectin antibody (anti-P+L Sel), or an anti-CD44 antibody prior to induction of eye inflammation (suture-induced inflammation).

FIG. 7 is a graph showing the percentage of rolling calcein-labeled dendritic cells in the limbal vessel compared to the total number of passing calcein-labeled dendritic cells in the limbal vessel in a mouse receiving no treatment or receiving an anti-VCAM-1 antibody (anti-VCAM-1), an anti-ICAM-1 antibody (anti-ICAM-1), or an anti-MadCAM-1 antibody.

FIG. 8 is a graph showing the percentage of rolling calcein-labeled dendritic cells in the limbal vessel compared to the total number of passing calcein-labeled dendritic cells in the limbal vessel in a mouse eye inflammation model receiving no treatment or receiving an anti-VCAM-1 antibody (anti VCAM-1), an anti-ICAM-1 antibody (anti ICAM-1), or an anti-MadCAM-1 antibody (anti MadCAM-1) prior to induction of eye inflammation (suture-induced inflammation).

FIG. 9 is a graph showing the percentage of calcein-labeled dendritic cells that stick to the limbal vessel for greater than 30 seconds compared to the total number of calcein-labeled dendritic cells passing in the limbal vessel in a mouse receiving no treatment or receiving an anti-VCAM-1 antibody (anti-VCAM-1), an anti-ICAM-1 antibody (anti-ICAM-1), an anti-MadCAM-1 antibody (anti-Mad-CAM), or PTX.

FIG. 10 is a graph showing the percentage of calcein-labeled dendritic cells that stick to the limbal vessel for greater than 30 seconds compared to the total number of calcein-labeled dendritic cells passing in the limbal vessel in a mouse eye inflammation model receiving no treatment or receiving an anti-VCAM-1 antibody (anti-VCAM-1 antibody), an anti-ICAM-1 antibody (anti-ICAM), an anti-MadCAM-1 antibody (anti-MadCAM), or PTX.

FIG. 11 is a graph showing the number of calcein-labeled dendritic cells present in the corneal epithelium, the corneal anterior stroma, or the corneal posterior stroma in a mouse untreated or treated with an anti-α4β7-antibody (a4b7), or in a mouse eye inflammation model that is untreated or treated with an anti-α4β7-antibody (a4b7) prior to induction of inflammation (suture-induced inflammation).

FIG. 12 is a graph showing the number of calcein-labeled dendritic cells present in the corneal epithelium, the corneal anterior stroma, or the corneal posterior stroma in a mouse treated with a control rat IgG or an anti-MadCAM-1-antibody (anti-MadCam), or in a mouse eye inflammation model that is treated with a control rat IgG or an anti-MadCam-antibody (anti-MadCAM) prior to induction of inflammation (suture-induced inflammation).

FIG. 13 is a graph showing the RT-PCR data for the expression of MadCAM-1 in the corneal limbal tissue of control mice (steady state mice) or mice having eye inflammation (suture-induced inflammation), or in Peyer's patches of control mice (steady state mice) (positive control).

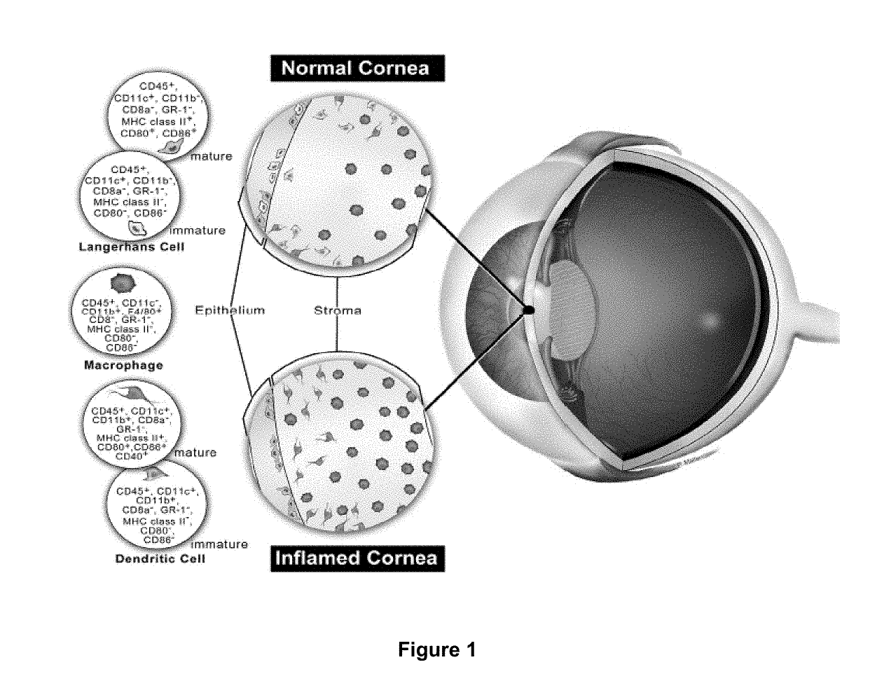

### DETAILED DESCRIPTION OF THE INVENTION

The invention is based, at least in part, on the discovery that antibodies that specifically bind to MadCAM-1, L-selectin, E-selectin, or α4β7 integrin decrease the migration of dendritic cells to the cornea in a mouse model of corneal inflammation. In view of this discovery, provided herein are methods for reducing corneal and/or conjunctival inflammation, reducing inflammatory cell (e.g., dendritic cell) recruitment to the cornea and/or the conjunctiva, and treating a corneal inflammatory disorder and/or a conjunctival inflammatory disorder in a subject that include administering one or more (e.g., two, three, four, or five) of a MadCAM-1 antagonist, an α4β7 integrin antagonist, a L-selectin antagonist, and a E-selectin antagonist to a subject. Also provided are compositions containing one or more (e.g., two, three, four, or five) of a MadCAM-1 antagonist, an α4β7 integrin antagonist, a L-selectin antagonist, and a S-selectin antagonist, as well as kits containing these compositions. Additional aspects and exemplary embodiments of these methods, compositions, and kits are described herein.

### Corneal Inflammation

Corneal inflammation is a condition that commonly results in the development of one or more symptoms in a subject. Non-limiting examples of such symptoms of corneal inflammation include: redness of the cornea; irritation, itchiness, burning, and/or dryness of the cornea; pain in the eye; excess tears or other discharge from an eye; difficulty opening an eyelid; blurred vision; sensitivity to light; and swelling around the eye. A subject can be diagnosed or identified as having corneal inflammation based on the observation or detection of one or more (e.g., at least two, three, or four) symptoms or physical characteristics of corneal inflammation selected from the group of: redness of the cornea; irritation, itchiness, burning, and/or dryness of the cornea; pain in the eye; excess tears or other discharge from an eye; difficulty opening an eyelid; blurred vision; sensitivity to light; swelling around the eye; and an elevated number of immunological cells present in the cornea (e.g., an elevated number of T-lymphocytes (e.g., effector T-cells), dendritic cells, stimulated monocytes, macrophages, B-cells, natural killer cells, eosinophils, and/or mast cells present in the cornea of the subject) (e.g., as compared to the same subject prior to the development of corneal inflammation, a control subject that does not have an eye disorder (a healthy subject), or a threshold value). In some embodiments, the detection of an elevated level of the number of immunological cells present in the cornea can be accomplished through the use of in vivo confocal microscopy using methods known in the art (see, e.g., the methods described in Cruzat et al., Semin. Ophthalmol. 25:171-177, 2010).

In some embodiments, the intensity, frequency, or duration of one or more symptoms of corneal inflammation can vary within the subject at any given time. For example, a subject having corneal inflammation can have one or more symptoms that are more prominent or more severe than other symptoms of corneal inflammation (depending on the subject and depending on the cause of the corneal inflammation).

In some embodiments, a subject having corneal inflammation (e.g., a low level of corneal inflammation) may only present with one or more symptoms that can only be detected using a microscopic technique (e.g., in vivo confocal microscopy) of the cornea. In some embodiments, a subject can present with both symptoms that can be detected without the use of a microscopic technique and symptoms that can only be detected using a microscopic technique. In some embodiments, a subject can be diagnosed or identified as having corneal inflammation by a medical professional (e.g., a physician, a physician's assistant, a nurse, a nurse's assistant, or a laboratory professional).

Corneal inflammation can be caused by a variety of factors. Non-limiting examples of causes of corneal inflammation include bacterial infection, fungal infection, parasite infection, viral infection (e.g., herpes simplex or herpes zoster), allergies, dry eye disorder, Fuchs' dystrophy, keratoconus, amyloidosis, lattice dystrophy, Stevens Johnson syndrome, physical corneal injury, Behcet's disease, contact lens wear, corneal graft rejection, dry eye syndrome, or immune keratitis (e.g., peripheral ulcerative keratitis). Additional causes of corneal inflammation are known in the art.

In some embodiments, the corneal inflammation is acute corneal inflammation. In some embodiments, the corneal inflammation is chronic corneal inflammation. In some embodiments, a subject having corneal inflammation has already been diagnosed as having a corneal inflammatory disorder (e.g., any of the corneal inflammatory disorders described herein). In some embodiments, the subject may already be receiving a treatment for corneal inflammation. In some embodiments, the subject may be resistant or show little responsiveness to a previous treatment for corneal inflammation. In some embodiments, the subject can be an infant, a child, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

### Corneal Inflammatory Disorders

Corneal inflammatory disorders are a family of disorders of the eye that are characterized by two of more of the following features in a subject: an elevated number of T-lymphocytes (e.g., effector T-cells) in a cornea, an elevated number of dendritic cells in a cornea, an elevated number of macrophages in a cornea, an elevated number of stimulated monocytes in a cornea, an elevated number of B-cells in a cornea, an elevated number of natural killer cells in a cornea, an elevated number of eosinophils in a cornea, an elevated number of mast cells in a cornea, an elevated level of redness in a cornea, pain in an eye, irritation, itchiness, burning, and/or dryness of a cornea, excess tears or other discharge from an eye, difficulty opening an eyelid, blurred vision, sensitivity to light, and swelling around an eye (e.g., as compared to levels in the same subject prior to development of corneal inflammation, a subject that does not have an eye disorder (a healthy subject), or a threshold value). Non-limiting examples of corneal inflammatory disorders include allergy, corneal abrasion, puncture, or trauma (including surgically-induced trauma), keratitis (e.g., both non-infectious and infectious keratitis), corneal autoimmune disease, or corneal allograft or xenograft rejection. In some embodiments, the corneal inflammatory disorder is infectious keratits (e.g., bacterial keratitis, fungal keratitis, viral keratitis, or parasitic keratitis). A non-limiting example of viral keratitis is herpes simplex keratitis or herpes zoster ophthalmicus. Additional examples of corneal inflammatory disorders are known in the art.

Methods for diagnosing a corneal inflammatory disorder in a subject are known in the art. For example, a subject can be diagnosed or identified as having a corneal inflammatory disorder by the observation or detection of one or more (e.g., at least two, three, or four) symptoms or physical characteristics selected from the group of: redness of the cornea; irritation, itchiness, burning, and/or dryness of the cornea; pain in the eye; excess tears or other discharge from an eye; difficulty opening an eyelid; blurred vision; sensitivity to light; swelling around the eye; and an elevated number of immunological cells present in the cornea (e.g., an elevated number of T-lymphocytes (e.g., effector T-cells, dendritic cells, macrophages, stimulated monocytes, B-cells, natural killer cells, eosinophils, and/or mast cells present in the cornea of the subject) (e.g., as compared to the levels in the same subject prior to the development of a corneal inflammatory disorder, a subject that does not have an eye disorder (a healthy subject), or a threshold value). In some embodiments, the detection of an elevated level of the number of immunological cells present in the cornea can be accomplished through the use of in vivo confocal microscopy using methods known in the art (see, e.g., the methods described in Cruzat et al., Semin. Ophthalmol. 25:171-177, 2010).

In some embodiments, the intensity, frequency, or duration of one or more symptoms of a corneal inflammatory disorder can vary within the subject at any given time. For example, a subject having a corneal inflammatory disorder can have one or more symptoms that are more prominent or more severe than other symptoms of a corneal inflammatory disorder (depending on the subject and depending on specific corneal inflammatory disorder).

In some embodiments, a subject having a corneal inflammatory disorder may only present with one or more symptoms that can only be detected using a microscopic technique (e.g., in vivo confocal microscopy) to visualize the cornea. In some embodiments, a subject can present with both symptoms that can be detected without the use of a microscopic technique and symptoms that can only be detected using a microscopic technique. In some embodiments, a subject can be diagnosed or identified as having a corneal inflammatory disorder by a medical professional (e.g., a physician, a physician's assistant, a nurse, a nurse's assistant, or a laboratory professional).

In some embodiments, a subject has already been diagnosed as having a corneal inflammatory disorder (e.g., any of the corneal inflammatory disorders described herein). In some embodiments, the subject may already be receiving a treatment for corneal inflammation. In some embodiments, the subject may be resistant or show little responsiveness to a previous treatment for corneal inflammation. In some embodiments, the subject can be an infant, a child, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

### Conjunctival Inflammation

Conjunctival inflammation is a condition that commonly results in the development of one or more symptoms in a subject. Non-limiting examples of such symptoms of conjunctival inflammation include: redness of the white of the eye or eyelid; irritation, itchiness, burning, and/or dryness of the eye; pain in the eye; excess tears or other discharge from an eye; difficulty opening an eyelid; blurred vision; sensitivity to light; and swelling around the eye. A subject can be diagnosed or identified as having conjunctival inflammation based on the observation or detection of one or more (e.g., at least two, three, or four) symptoms or physical characteristics of conjunctival inflammation selected from the group of: redness of the white of the eye or eyelid; irritation, itchiness, burning, and/or dryness of the eye; pain in the eye; excess tears or other discharge from an eye; difficulty opening an eyelid; blurred vision; sensitivity to light; swelling around the eye; and an elevated number of immunological cells present in the conjunctiva (e.g., an elevated number of T-lymphocytes (e.g., effector T-cells), dendritic cells, stimulated monocytes, macrophages, B-cells, natural killer cells, eosinophils, and/or mast cells present in the conjunctiva of the subject) (e.g., as compared to the same subject prior to the development of conjunctival inflammation, a control subject that does not have an eye disorder (a healthy subject), or a threshold value). In some embodiments, the detection of an elevated level of the number of immunological cells present in the conjunctiva can be accomplished through the use of in vivo confocal microscopy using methods known in the art (see, e.g., the methods described in Cruzat et al., Semin. Ophthalmol. 25:171-177, 2010).

In some embodiments, the intensity, frequency, or duration of one or more symptoms of conjunctival inflammation can vary within the subject at any given time. For example, a subject having conjunctival inflammation can have one or more symptoms that are more prominent or more severe than other symptoms of conjunctival inflammation (depending on the subject and depending on the cause of the conjunctival inflammation).

In some embodiments, a subject having conjunctival inflammation (e.g., a low level of conjunctival inflammation) may only present with one or more symptoms that can only be detected using a microscopic technique (e.g., in vivo confocal microscopy) of the conjunctiva. In some embodiments, a subject can present with both symptoms that can be detected without the use of a microscopic technique and symptoms that can only be detected using a microscopic technique. In some embodiments, a subject can be diagnosed or identified as having conjunctival inflammation by a medical professional (e.g., a physician, a physician's assistant, a nurse, a nurse's assistant, or a laboratory professional).

Conjunctival inflammation can be caused by a variety of factors. Non-limiting examples causes of conjunctival inflammation include viruses, bacteria (e.g., gonorrhea or chlamydia), irritants (e.g., shampoos, dirt, smoke, or chlorine), an allergen, or contact lens wear. Additional causes of conjunctival inflammation are known in the art.

In some embodiments, the conjunctival inflammation is acute conjunctival inflammation. In some embodiments, the conjunctival inflammation is chronic conjunctival inflammation. In some embodiments, a subject having conjunctival inflammation has already been diagnosed as having a conjunctival inflammatory disorder (e.g., any of the conjunctival inflammatory disorders described herein). In some embodiments, the subject may already be receiving a treatment for conjuctival inflammation. In some embodiments, the subject may be resistant or show little responsiveness to a previous treatment for conjunctival inflammation. In some embodiments, the subject can be an infant, a child, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

### Conjunctival Inflammatory Disorders

Conjunctival inflammatory disorders are a family of disorders of the eye that are characterized by two of more of the following features in a subject: an elevated number of T-lymphocytes (e.g., effector T-cells) in a conjunctiva, an elevated number of dendritic cells in a conjunctiva, an elevated number of macrophages in a conjunctiva, an elevated number of stimulated monocytes in a conjunctiva, an elevated number of B-cells in a conjunctiva, an elevated number of natural killer cells in a conjunctiva, an elevated number of eosinophils in a conjunctiva, an elevated number of mast cells in a conjunctiva, an elevated level of redness in the white of an eye or an eyelid, pain in an eye, irritation, itchiness, burning, and/or dryness of an eye, excess tears or other discharge from an eye, difficulty opening an eyelid, blurred vision, sensitivity to light, and swelling around an eye (e.g., as compared to levels in the same subject prior to development of conjunctival inflammation, a subject that does not have an eye disorder (a healthy subject), or a threshold value). Non-limiting examples of conjunctival inflammatory disorders include viral conjunctivitis, bacterial conjunctivis, fungal conjunctivitis, parasitic conjunctivitis, or allergic conjunctivitis. Additional examples of conjunctival inflammatory disorders are known in the art.

Methods for diagnosing a conjunctival inflammatory disorder in a subject are known in the art. For example, a subject can be diagnosed or identified as having a conjunctival inflammatory disorder by the observation or detection of one or more (e.g., at least two, three, or four) symptoms or physical characteristics selected from the group of: redness of the cornea; irritation, itchiness, burning, and/or dryness of an eye; pain in the eye; excess tears or other discharge from an eye; difficulty opening an eyelid; blurred vision; sensitivity to light; swelling around the eye; and an elevated number of immunological cells present in the conjunctiva (e.g., an elevated number of T-lymphocytes (e.g., effector T-cells, dendritic cells, macrophages, stimulated monocytes, B-cells, natural killer cells, eosinophils, and/or mast cells present in the conjunctiva of the subject) (e.g., as compared to the levels in the same subject prior to the development of a conjunctival inflammatory disorder, a subject that does not have an eye disorder (a healthy subject), or a threshold value). In some embodiments, the detection of an elevated level of the number of immunological cells present in the conjunctiva can be accomplished through the use of in vivo confocal microscopy using methods known in the art (see, e.g., the methods described in Cruzat et al., Semin. Ophthalmol. 25:171-177, 2010).

In some embodiments, the intensity, frequency, or duration of one or more symptoms of a conjunctival inflammatory disorder can vary within the subject at any given time. For example, a subject having a conjuctival inflammatory disorder can have one or more symptoms that are more prominent or more severe than other symptoms of a conjunctival inflammatory disorder (depending on the subject and depending on specific conjunctival inflammatory disorder).

In some embodiments, a subject having a conjunctival inflammatory disorder may only present with one or more symptoms that can only be detected using a microscopic technique (e.g., in vivo confocal microscopy) to visualize the conjunctiva. In some embodiments, a subject can present with both symptoms that can be detected without the use of a microscopic technique and symptoms that can only be detected using a microscopic technique. In some embodiments, a subject can be diagnosed or identified as having a conjunctival inflammatory disorder by a medical professional (e.g., a physician, a physician's assistant, a nurse, a nurse's assistant, or a laboratory professional).

In some embodiments, a subject has already been diagnosed as having a conjunctival inflammatory disorder (e.g., any of the conjunctival inflammatory disorders described herein). In some embodiments, the subject may already be receiving a treatment for conjunctival inflammation. In some embodiments, the subject may be resistant or show little responsiveness to a previous treatment for conjunctival inflammation. In some embodiments, the subject can be an infant, a child, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

### Antagonists

Provided herein, in part, are methods for decreasing corneal and/or conjunctival inflammation, decreasing immunological cell (e.g., dendritic cell) migration to the cornea and/or conjunctiva, and treating a corneal inflammatory disorder and/or conjunctival inflammatory disorder that include the administration of one or more of a MadCAM-1 antagonist, an α4β7 integrin antagonist, a L-selectin antagonist, and a E-selectin antagonist to a subject.

Mucosal vascular addressin cell adhesion molecule 1 (MadCAM-1), also known as addressin, binds to α4β7 integrin, L-selectin, and α4β1 integrin. Mature human MadCAM-1 binds to α4β7 integrin through its two conserved immunoglobulin superfamily (IgSF) domains (see, e.g., Tan et al., Structure 15:793-801, 1998). Conserved residues in human MadCAM-1 that are important for integrin binding (e.g., α4β7 and α4β1 integrin) include the CD loop of the first IgSF domain and amino acid residues Asp42 and Arg70 (see, Tan et al., supra). In contrast, the amino acids that are glycosylated in MadCAM-1 are important for L-selectin binding (see, Tan et al., supra). A precursor form of MadCAM-1 is processed to remove a signal peptide. The mature form of MadCAM-1 contains a short cytoplasmic domain, a transmembrane domain, a mucin-like domain, and the two IgSF domains (see, Leung et al., Immunol. Cell. Biol. 74:490-496, 1996; Shyjan et al., J. Immunol. 156:2851-2857, 1996). Non-limiting examples of human mature, processed forms of MadCAM-1 protein are SEQ ID NOs: 1-7.

Non-limiting examples of MadCAM-1 antagonists include antibodies and antigen-binding antibody fragments that specifically bind to MadCAM-1. Additional non-limiting examples of MadCAM-1 antagonists include soluble α4β7 agents, soluble α4β1 agents, and soluble L-selectin molecules. Exemplary embodiments and aspects of these MadCAM-1 antagonists are described below.

Alpha-4 beta-7 integrin is a heterodimer of the α4 integrin and the β74 integrin that is constitutively expressed on the surface of leukocytes including lymphocytes, monocytes, eosinophils and basophils (see Hemler et al., J. Biol. Chem. 262:11478-11485, 1987; and Bochner et al., J. Exp. Med. 173:1553-1556, 1991). A non-limiting example of the sequence of human mature, processed α4 integrin is SEQ ID NO: 8, and a non-limiting example of the sequence of human mature, process β7 integrin is SEQ ID NO: 9.

Non-limiting examples of α4β7 integrin antagonists include antibodies and antigen-binding antibody fragments that specifically bind to α4β7 integrin. Additional non-limiting examples of α4β7 integrin antagonists include soluble MadCAM-1 molecules, small molecule α4β7 integrin antagonists, and antibodies or antigen-binding fragments that bind to α4 integrin or β7 integrin. Exemplary embodiments and aspects of these α4β7 antagonists are described below.

L-selectin, also known as CD62L, is a cell adhesion molecule expressed in the plasma membrane of leukocytes. It contains several domains, including a signal sequence, transmembrane domain, and cytoplasmic domain. L-selectin binds to GlyCAM-1, CD34, MadCAM-1, and PSGL-1. A non-limiting example of the sequence of human mature, processed L-selectin is SEQ ID NO: 19.

Non-limiting examples of L-selectin antagonists are antibodies or an antigen-binding antibody fragments that specifically bind to L-selectin, or a soluble MadCAM-1 agent, a soluble CD34 molecule, a soluble PSGL-1 molecule, or a soluble GlyCAM-1 agent. Additional examples of L-selectin antagonists are small molecules (e.g., the small molecule L-selectin antagonists described herein or known in the art).

E-selectin, also known as CD62E, endothelial-leukocyte adhesion molecule 1 (ELAM-1), or leukocyte-endothelial cell adhesion molecule 2 (LECAM2), is a cell adhesion molecule expressed on endothelial cells (e.g., endothelial cells activated by cytokines). It contains several domains, including a signal sequence, transmembrane domain, and cytoplasmic domain. E-selectin binds to sialylated carbohydrates (e.g., carbohydrates of the Lewis X and Lewis A families) present on proteins expressed by leukocytes (e.g., monocytes, granulocytes, and T-lymphocytes).

Non-limiting examples of E-selectin antagonists are antibodies or an antigen-binding antibody fragments that specifically bind to E-selectin. Additional examples of E-selectin antagonists are small molecules (e.g., the small molecule E-selectin antagonists described herein or known in the art).

### Antibodies and Antigen-Binding Antibody Fragments

Antibodies that specifically bind to MadCAM-1, α4β7, α4 integrin, β7 integrin, L-selectin, or E-selectin can be, for example, polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG₁, IgG₂, IgG₃, IgG₄, IgA₁, and IgA₂), or subclass. In some embodiments, the antibody or antigen-binding fragment thereof is an IgG₁ antibody or antigen-binding fragment thereof. In other embodiments, the antibody or antigen-binding fragment thereof is an IgG4 antibody or antigen-binding fragment thereof. Immunoglobulins may have both a heavy and light chain.

Antibodies and antibody fragments as referred to herein include variants (including derivatives and conjugates) of antibodies or antibody fragments and multi-specific (e.g., bi-specific) antibodies or antibody fragments. Examples of antibodies and antigen-binding fragments thereof include, but are not limited to: single-chain Fvs (scFvs), Fab fragments, Fab′ fragments, F(ab′)2, disulfide-linked Fvs (sdFvs), Fvs, and fragments containing either a VL or a VH domain. The term “single chain Fv” or “scFv” as used herein refers to a polypeptide comprising at least one VL domain of an antibody linked to at least one VH domain of an antibody.

An isolated MadCAM-1, α4β7, α4 integrin, β7 integrin, L-selectin, or E-selectin, or a fragment thereof (e.g., at least 7, 10, 15, 20, 25, 30, 35, 40, 50, 60, 70, 80, 90, or 100 amino acids of a wild type form of MadCAM-1, α4 integrin, β7 integrin, α4β7 integrin, L-selectin, or E-selectin) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to a protein that is immunogenic in the species to be immunized, e.g., keyhole limpet hemocyanin, serum albumin, bovine thyroglobulin, or soybean trypsin inhibitor using a bifunctional or derivitizing agent, for example, malimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals can be injected with the antigenic peptide or protein more than one time (e.g., twice, three times, or four times).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of MadCAM-1, such as the soluble MadCAM-1 molecules described herein, a fragment of α4β7 integrin, such as the soluble α4β7 agents described herein, a fragment of α4 integrin, a fragment of β7 integrin, a fragment of L-selectin, such as the soluble L-selectin agents described herein, or a fragment of E-selectin). The preparation can further include an adjuvant, such as Freund's complete or incomplete adjuvant, or a similar immunostimulatory agent.

Polyclonal antibodies can be prepared as described above by immunizing a suitable subject with MadCAM-1, α4 integrin, β7 integrin, α4β7 integrin, L-selectin, or E-selectin, or an antigenic peptide thereof (e.g., a fragment of MadCAM-1, such as the soluble MadCAM-1 molecules described herein, a fragment of α4β7 integrin, such as the soluble α4β7 agents described herein, a fragment of α4 integrin, a fragment of β7 integrin, a fragment of L-selectin, such as the soluble L-selectin molecules described herein, or a fragment of E-selectin) as an immunogen. The antibody titer in the immunized subject can be monitored over time by standard techniques, such as with an enzyme-linked immunosorbent assay (ELISA) using the immobilized MadCAM-1, α4β7, α4 integrin, β7 integrin, L-selectin, or E-selectin, or fragment thereof. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, N.Y.). Hybridoma cells producing a monoclonal antibody are detected by screening the hybridoma culture supernatants for antibodies that bind the polypeptide or epitope of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide or a peptide fragment containing the epitope of interest. Kits for generating and screening phage display libraries are commercially available (e.g., the Pharmacia Recombinant Phage Antibody System, Catalog No. 27-9400-01; and the Stratagene SurfZAP* Phage Display Kit, Catalog No. 240612). Additionally, examples of methods and reagents particularly amenable for use in generating and screening an antibody display library can be found in, for example, U.S. Pat. No. 5,223,409; WO 92/18619; WO 91/17271; WO 92/2079; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; and Griffiths et al., FHBO J. 12:725-734, 1993.

In some embodiments of any of the methods described herein, the antibodies or antigen-binding fragments are human antibodies, humanized antibodies, or chimeric antibodies that contain a sequence from a human antibody (e.g., a human immunoglobulin constant domain or human immunoglobulin variable domain framework regions). Humanized antibodies are chimeric antibodies that contain a minimal sequence derived from non-human (e.g., mouse) immunoglobulin. In some embodiments, a humanized antibody is a human antibody that has been engineered to contain at least one complementary determining region (CDR) present in a non-human antibody (e.g., a mouse, rat, rabbit, or goat antibody). In some embodiments, a humanized antibody or fragment thereof can contain all three CDRs of a light chain and/or a heavy chain of a non-human antibody that specifically binds to MadCAM-1 or a fragment thereof, a non-human antibody that specifically binds to α4β7 or a fragment thereof, a non-human antibody that specifically binds to L-selectin or a fragment thereof, or a non-human antibody that specifically binds to E-selectin or a fragment thereof. In some embodiments, the framework region residues of the human immunoglobulin are replaced by corresponding non-human (e.g., mouse) antibody residues. In some embodiments, the humanized antibodies can contain residues which are not found in the human antibody or in the non-human (e.g., mouse) antibody. Methods for making a humanized antibody from a non-human (e.g., mouse) monoclonal antibody are known in the art. Additional non-limiting examples of making a chimeric (e.g., humanized) antibody are described herein.

In some embodiments, the antibodies are chimeric antibodies that contain a light chain immunoglobulin that contains the light chain variable domain of a non-human antibody (e.g., a mouse antibody) or at least one CDR of a light chain variable domain of a non-human antibody (e.g., a mouse antibody) and the constant domain of a human immunoglobulin light chain (e.g., human κ chain constant domain). In some embodiments, the antibodies are chimeric antibodies that contain a heavy chain immunoglobulin that contains the heavy chain variable domain of a non-human (e.g., a mouse antibody) or at least one CDR of a heavy chain variable domain of a non-human (e.g., a mouse antibody) and the constant domain of a human immunoglobulin heavy chain (e.g., a human IgG heavy chain constant domain). In some embodiments, the chimeric antibodies contain a portion of a constant (Fc domain) of a human immunoglobulin.

In some embodiments, the antibodies or antigen-binding fragments thereof can be multi-specific (e.g., multimeric). For example, the antibodies can take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers. Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) (available, for example, from Pierce Biotechnology, Inc., Rockford, Ill.) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers (see, for example, WO 96/27011).

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents are well known in the art and are disclosed in U.S. Pat. No. 4,676,980, along with a variety of cross-linking techniques.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Additional methods have been developed to facilitate the direct recovery of Fab′-SH fragments from E. coli, which can be chemically coupled to form bi-specific antibodies. Shalaby et al. (J. Exp. Med 175:217-225, 1992) describes the production of a fully-humanized bi-specific antibody F(ab′)2 molecule. Each Fab′ fragment was separately secreted from E. coli and subjected to direct chemical coupling in vitro to form the bi-specific antibody.

Additional techniques for making and isolating bi-specific antibody fragments directly from recombinant cell culture have also been described. For example, bi-specific antibodies have been produced using leucine zippers (Kostelny et al., J. Immunol. 148:1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

The diabody technology described by Hollinger et al. (Proc. Natl. Acad. Sci. U.S.A. 90:6444-6448, 1993) is an additional method for making bi-specific antibody fragments. The fragments contain a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another method for making bi-specific antibody fragments by the use of single-chain Fv (sFv) dimers has been described in Gruber et al. (J. Immunol. 153:5368, 1994). Alternatively, the bi-specific antibody can be a “linear” or “single-chain antibody” produced using the methods described, for example, in Zapata et al. (Protein Eng. 8:1057-1062, 1995). In some embodiments the antibodies have more than two antigen-binding sites. For example, tri-specific antibodies can be prepared as described in Tutt et al. (J. Immunol. 147:60, 1991).

Alternatively, antibodies can be made to multimerize through recombinant DNA techniques. IgM and IgA naturally form antibody multimers through the interaction with the mature J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules (see, for example, Chintalacharuvu et al., Clin. Immunol. 101:21-31, 2001, and Frigerio et al., Plant Physiol. 123:1483-1494, 2000). IgA dimers are naturally secreted into the lumen of mucosa-lined organs. This secretion is mediated through the interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (Johansen et al., J. Immunol., 167:5185-192, 2001). ScFv dimers can also be formed through recombinant techniques known in the art. An example of the construction of scFv dimers is given in Goel et al. (Cancer Res. 60:6964-71, 2000). Antibody multimers may be purified using any suitable method known in the art, including, but not limited to, size exclusion chromatography.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

Any of the antibodies or antigen-binding fragments described herein may be conjugated to a label (e.g., a radioisotope, fluorophore, chromophore, or the like) or a therapeutic agent (e.g., a proteinaceous or small molecule therapeutic agent).

In some embodiments of any of the methods described herein, the antibody or antigen-binding fragment thereof binds to an epitope on MadCAM-1, α4β7, α4 integrin, or β7 integrin with an KD equal to or less than 1×10⁻⁷ M, a KD equal to or less than 1×10⁻⁸ M, a KD equal to or less than 5×10⁻⁸ M, a KD equal to or less than 5×10⁻⁹M, or a KD equal to or less than 1×10⁻⁹M under physiological conditions (e.g., in phosphate buffered saline).

Methods for detecting the ability of an antibody or antigen-binding antibody fragment to bind to MadCAM-1, α4β7, α4 integrin, β7 integrin, L-selectin, or E-selectin can be performed using methods known in the art, e.g., surface plasmon resonance (SPR).

Additional exemplary antibodies that specifically bind to MadCAM-1 are known in the art (e.g., U.S. Patent Application Publication No. 20090214558; herein incorporated by reference; and MadCAM-1 antibodies commercially available from Abbiotec, Santa Cruz Technology, and Hycult Biotech). Additional exemplary antibodies that specifically bind to α4β7 are known in the art (e.g., natalizumab, vedolizumab, and the humanized antibodies described in U.S. Pat. Nos. 5,840,299; 6,602,503; 7,482,003; and 7,618,630; and U.S. Patent Applications Nos. 2010/0203042 and 2009/0202527 (each listed patent and patent application is incorporated herein by reference). In some embodiments, an α4β7 antagonist is an antibody or an antigen-binding fragment thereof that can bind to α4 and/or β7. An exemplary humanized antibody that binds to human α4 integrin is natalizumab. Additional examples of humanized antibodies that bind to human α4 integrin are described in U.S. Patent Application Publication Nos. 2010/0081793. An exemplary humanized antibody that binds to human β7 integrin is rhuMAb Beta7.

Additional exemplary antibodies that specifically bind to L-selectin are known in the art (e.g., aselizumab, HuDREG-55, HuEP5C7, and DREG-200). Additional exemplary antibodies that specifically bind to E-selectin are known in the art (e.g., HuEP5C7, CDP850, and CDP-850).

In some embodiments, the antibodies or antigen-binding fragments described herein are isolated or purified (e.g., at least 70%, 75%, 80%, 85%, 90%, 96%, 97%, 98%, or 99% pure by dry weight).

Soluble L-Selectin Molecules

The mature form of L-selectin contains the following domains: a lectin domain, an epiderminal growth factor domain, two SCR domains, a transmembrane domain and a cytoplasmic domain. A soluble L-selectin molecule contains an amino acid sequence that is at least 95% (e.g., 96%, 97%, 98%, 99%, or 100%) identical to a contiguous sequence (e.g., at least 50, 75, 100, 125, 150, 175, or 200 amino acids) in a wild type form of L-selectin (e.g., a precursor or processed L-selectin, e.g., any one of SEQ ID NOS: 10 and 19) that is soluble at a physiological pH and has the ability to specifically bind to MadCAM-1. In some embodiments, the soluble L-selectin lacks one or more (e.g., two, three, or four of its domains) of its domains. In some embodiments, the soluble L-selectin molecule lacks its signal sequence, its transmembrane domain, and its cytoplasmic domain. In some embodiments, the soluble L-selectin molecule contains an additional amino acid sequence (e.g., a sequence that stabilizes the protein or increases the protein's half-life in vivo, e.g., an Fc domain or serum albumin).

In some embodiments, the soluble L-selectin molecule contains a sequence that is at least 95% (e.g., at least 96%, 97%, 98%, 99%, or 100%) identical to a contiguous sequence (e.g., at least 50, 75, 100, 125, 150, 175, or 200 amino acids) present between amino acids 171 and amino acid 385 in SEQ ID NO: 10 shown below. The mRNA encoding precursor L-selectin is also shown below. Soluble L-selectin molecules can be generated using molecular biology skills known in the art. The ability of a soluble L-selectin molecule to bind to MadCAM-1, can be performed using cell binding assays (e.g., cells expressing one or more of MadCAM-1), competitive binding assays with antibodies that specifically bind to MadCAM-1, or surface plasmon resonance (SPR).

Precursor human L-selectin polypeptide (SEQ ID NO: 10) with the signal peptide underlined and the transmembrane domain shown in bold.

(SEQ ID NO: 10)
MGCRRTREGP SKAMIFPWKC QSTQRDLWNI FKLWGWTMLC
CDFLAHHGTD CWTYHYSEKP MNWQRARRFC RDNYTDLVAI
QNKAEIEYLE KTLPFSRSYY WIGIRKIGGI WTWVGTNKSL
TEEAENWGDG EPNNKKNKED CVEIYIKRNK DAGKWNDDAC
HKLKAALCYT ASCQPWSCSG HGECVEIINN YTCNCDVGYY
GPQCQFVIQC EPLEAPELGT MDCTHPLGNF SFSSQCAFSC
SEGINLIGIE ETTCGPFGNW SSPEPTCQVI QCEPLSAPDL
GIMNCSHPLA SFSFTSACTF ICSEGTELIG KKKTICESSG
IWSNPSPICQ KLDKSFSMIK EGDYNPLFIP VAVMVTAFSG
LAFIIWLARR LKKGKKSKRS MNDPY

An exemplary cDNA encoding precursor human L-selectin is SEQ ID NO: 15.

Soluble PSGL-1 Agents

The precursor form of PSGL-1 contains a signal sequence that is cleaved to form the mature protein. A soluble PSGL-1 molecule contains an amino acid sequence that is at least 95% (e.g., at least 96%, 97%, 98%, 99%, or 100%) identical to a contiguous sequence (e.g., at least 50, 75, 100, 125, 150, 175, 200, 225, 250, 275, or 300 amino acids) in a wild type form of PSGL-1 (e.g., a precursor or processed PSGL-1, e.g., SEQ ID NO: 21) that is soluble at a physiological pH and has the ability to specifically bind to L-selectin. In some embodiments, the soluble PSGL-1 lacks one or more (e.g., two, three, or four of its domains) of its domains. In some embodiments, the soluble PSGL-1 molecule lacks its signal sequence, its transmembrane domain, and its cytoplasmic domain. In some embodiments, the soluble PSGL-1 molecule contains an additional amino acid sequence (e.g., a sequence that stabilizes the protein or increases the protein's half-life in vivo, e.g., an Fc domain or serum albumin).

In some embodiments, the soluble PSGL-1 molecule contains a sequence that is at least 95% (e.g., 96%, 97%, 98%, 99%, or 100%) identical to a contiguous sequence (e.g., at least 50, 75, 100, 125, 150, 175, 200, 225, 250, 275, or 300 amino acids) present between amino acids 18 and amino acid 320 in SEQ ID NO: 21 shown below. The mRNA encoding precursor PSGL-1 is also shown below. Soluble PSGL-1 molecules can be generated using molecular biology skills known in the art. The ability of a soluble PSGL-1 molecule to specifically bind to L-selectin, can be performed using cell binding assays (e.g., cells expressing L-selectin), competitive binding assays with antibodies that specifically bind to L-selectin, or surface plasmon resonance (SPR).

Precursor human PSGL-1 polypeptide (SEQ ID NO: 21) with the signal peptide underlined and the transmembrane domain shown in bold.

| (SEQ ID NO: 21) | |
|---|---|
| 1 | MPLQLLLLLI LLGPGNSLQL WDTWADEAEK ALGPLLARDR RQATEYEYLD YDFLPETEPP |
| 61 | EMLRNSTDTT PLTGPGTPES TTVEPAARRS TGLDAGGAVT ELTTELANMG NLSTDSAAME |
| 121 | IQTTQPAATE AQTTQPVPTE AQTTPLAATE AQTTRLTATE AQTTPLAATE AQTTPPAATE |
| 181 | AQTTQPTGLE AQTTAPAAME AQTTAPAAME AQTTPPAAME AQTTQTTAME AQTTAPEATE |
| 241 | AQTTQPTATE AQTTPLAAME ALSTEPSATE ALSMEPTTKR GLFIPFSVSS VTHKGIPMAA |
| 301 | SNLSVNYPVG APDHISVKQC LLAILILALV ATIFFVCTVV LAVRLSRKGH MYPVRNYSPT |
| 361 | EMVCISSLLP DGGEGPSATA NGGLSKAKSP GLTPEPREDR EGDDLTLHSF LP |

An exemplary cDNA encoding precursor human PSGL-1 is SEQ ID NO: 22.

Soluble CD34 Molecules

The precursor form of CD34 contains a signal sequence that is cleaved to form the mature protein. A soluble CD34 molecule contains an amino acid sequence that is at least 95% (e.g., 96%, 97%, 98%, 99%, or 100%) identical to a contiguous sequence (e.g., at least 50, 75, 100, 125, 150, 175, 200, or 250 amino acids) in a wild type form of CD34 (e.g., a precursor or processed CD34, e.g., SEQ ID NO: 23) that is soluble at a physiological pH and has the ability to specifically bind to L-selectin. In some embodiments, the soluble CD34 lacks one or more (e.g., two, three, or four of its domains) of its domains. In some embodiments, the soluble CD34 molecule lacks its signal sequence, its transmembrane domain, and its cytoplasmic domain. In some embodiments, the soluble CD34 molecule contains an additional amino acid sequence (e.g., a sequence that stabilizes the protein or increases the protein's half-life in vivo, e.g., an Fc domain or serum albumin).

In some embodiments, the soluble CD34 molecule contains a sequence that is at least 95% (e.g., 96%, 97%, 98%, 99%, or 100%) identical to a contiguous sequence (e.g., at least 50, 75, 100, 125, 150, 175, 200, or 250 amino acids) present between amino acids 32 and amino acid 290 in SEQ ID NO: 23 shown below. The mRNA encoding precursor CD34 is also shown below. Soluble CD34 molecules can be generated using molecular biology skills known in the art. The ability of a soluble CD34 molecule to bind to L-selectin, can be performed using cell binding assays (e.g., cells expressing L-selectin), competitive binding assays with antibodies that specifically bind to L-selectin, or surface plasmon resonance (SPR).

Precursor human CD34 polypeptide (SEQ ID NO: 23) with the signal peptide underlined and the transmembrane domain shown in bold.

| (SEQ ID NO: 23) | |
|---|---|
| 1 | MLVRRGARAG PRMPRGWTAL CLLSLLPSGF MSLDNNGTAT PELPTQGTFS NVSTNVSYQE |
| 61 | TTTPSTLGST SLHPVSQHGN EATTNITETT VKFTSTSVIT SVYGNTNSSV QSQTSVISTV |
| 121 | FTTPANVSTP ETTLKPSLSP GNVSDLSTTS TSLATSPTKP YTSSSPILSD IKAEIKCSGI |
| 181 | REVKLTQGIC LEQNKTSSCA EFKKDRGEGL ARVLCGEEQA DADAGAQVCS LLLAQSEVRP |
| 241 | QCLLLVLANR TEISSKLQLM KKHQSDLKKL GILDFTEQDV ASHQSYSQKT LIALVTSGAL |
| 301 | LAVLGITGYF LMNRRSWSPT GERLGEDPYY TENGGGQGYS SGPGTSPEAQ GKASVNRGAQ |
| 361 | ENGTGQATSR NGHSARQHVV ADTEL |

An exemplary cDNA encoding precursor human CD34 is SEQ ID NO: 24.

### Soluble GlyCAM-1 Molecules

The precursor form of GlyCAM-1 contains a signal sequence that is cleaved to form the mature protein. A soluble GlyCAM-1 molecule contains an amino acid sequence that is at least 95% (e.g., 96%, 97%, 98%, 99%, or 100%) identical to a contiguous sequence (e.g., at least 10 or 20 amino acids) in a wild type form of GlyCAM-1 (e.g., a precursor or processed GlyCAM-1, e.g., SEQ ID NO: 25) that is soluble at a physiological pH and has the ability to specifically bind to L-selectin. In some embodiments, the soluble GlyCAM-1 molecule lacks its signal sequence. In some embodiments, the soluble GlyCAM-1 molecule contains an additional amino acid sequence (e.g., a sequence that stabilizes the protein or increases the protein's half-life in vivo, e.g., an Fc domain or serum albumin).

In some embodiments, the soluble GlyCAM-1 molecule contains a sequence that is at least 95% (e.g., at least 96%, 97%, 98%, 99%, or 100%) identical to a contiguous sequence (e.g., at least 10 or 20 amino acids) present between amino acids 19 and amino acid 47 in SEQ ID NO: 25 shown below. The mRNA encoding precursor GlyCAM-1 is also shown below. Soluble GlyCAM-1 molecules can be generated using molecular biology skills known in the art. The ability of a soluble GlyCAM-1 molecule to bind to L-selectin, can be performed using cell binding assays (e.g., cells expressing L-selectin), competitive binding assays with antibodies that specifically bind to L-selectin, or surface plasmon resonance (SPR).

Precursor human GlyCAM-1 polypeptide (SEQ ID NO: 25) with the signal peptide underlined.

| (SEQ ID NO: 25) | |
|---|---|
| 1 | MKFFMVLLPA SLASTSLAIL DVESGLLPQL SVLLSNRLRG KTCQTGP |

An exemplary cDNA encoding precursor human GlyCAM-1 is SEQ ID NO: 26.

Soluble α4β7 Agents

Alpha-4, beta-7 integrin is a heterodimer that is composed of α4 integrin and β7 integrin. Both the α4 integrin and the β4 integrin contain a number of domains including a signal sequence, a transmembrane domain, and a cytoplasmic domain. A soluble α4β7 agent is a composition that contains a protein (e.g., a single polypeptide or heterodimeric protein) containing an amino acid sequence that is at least 95% (e.g., at least 96%, 97%, 98%, 99%, or 100%) identical to a contiguous sequence (e.g., at least 50, 75, 100, 125, 150, 175, 200, 250, 300, 350, 400, 450, 500, 550, 600, 650, 700, 750, 800, or 850 amino acids) in a wild type form of α4 integrin (e.g., precursor or processed α4 integrin, e.g., any one of SEQ ID NOS: 8 and 12) and an amino acid sequence that is at least 95% (e.g., at least 96%, 97%, 98%, 99%, or 100%) identical to a contiguous sequence (e.g., at least 50, 75, 100, 125, 150, 175, 200, 250, 300, 350, 400, 450, 500, 550, 600, 650, or 700 amino acids) in a wild type form of β7 integrin (e.g., precursor or processed β7 integrin, e.g., any one of SEQ ID NOS: 9 and 13) that is soluble at physiological pH and has the ability to specifically bind to MadCAM-1. In some embodiments, the amino acid sequence that is at least 95% identical to a contiguous sequence in α4 integrin lacks the signal sequence, the transmembrane domain, and cytoplasmic domain of α4 integrin, and/or the amino acid sequence that is at least 95% identical to a contiguous sequence in β7 integrin lacks the signal sequence, the transmembrane domain, and cytoplasmic domain of β7 integrin. In some embodiments, the soluble α4β7 agent contains a polypeptide that contains an additional amino acid sequence (e.g., a sequence that stabilizes the polypeptide or increases the polypeptide's half-life in vivo, e.g., an Fc domain or bovine serum albumin).

In some embodiments, the amino acid sequence that is at least 95% identical to a contiguous sequence in α4 integrin contains a sequence that is at least 95% (e.g., 96%, 97%, 98%, 99%, or 100%) identical to a contiguous sequence (e.g., at least 50, 75, 100, 125, 150, 175, 200, 250, 300, 350, 400, 450, 500, 550, 600, 650, 700, 750, 800, or 850 amino acids) present between amino acids 34 and amino acid 977 in SEQ ID NO: 12. In some embodiments, the amino acid sequence that is at least 95% identical to a contiguous sequence in β7 integrin contains a sequence that is at least 95% identical (e.g., at least 96%, 97%, 98%, 99%, or 100% identical) to a contiguous sequence (e.g., at least 50, 75, 100, 125, 150, 175, 200, 250, 300, 350, 400, 450, 500, 550, 600, 650, or 700 amino acids) present between amino acids 20 and amino acid 723 in SEQ ID NO: 13. The mRNA encoding α4 and the mRNA encoding β7 are also shown below. Soluble α4β7 agents can be generated using molecular biology skills known in the art. The ability of a soluble α4β7 agent to bind to MadCAM-1, can be performed using cell binding assays (e.g., cells expressing one or more of MadCAM-1), competitive binding assays with antibodies that specifically bind to MadCAM-1, or surface plasmon resonance (SPR).

Precursor human α4 integrin polypeptide (SEQ ID NO: 12) with the signal sequence underlined and the transmembrane domain shown in bold.

| (SEQ ID NO: 12) | |
|---|---|
| 1 | MAWEARREPG PRRAAVRETV MLLLCLGVPT GRPYNVDTES ALLYQGPHNT LFGYSVVLHS |
| 61 | HGANRWLLVG APTANWLANA SVINPGAIYR CRIGKNPGQT CEQLQLGSPN GEPCGKTCLE |
| 121 | ERDNQWLGVT LSRQPGENGS IVTCGHRWKN IFYIKNENKL PTGGCYGVPP DLRTELSKRI |
| 181 | APCYQDYVKK FGENFASCQA GISSFYTKDL IVMGAPGSSY WTGSLFVYNI TTNKYKAFLD |
| 241 | KQNQVKFGSY LGYSVGAGHF RSQHTTEVVG GAPQHEQIGK AYIFSIDEKE LNILHEMKGK |
| 301 | KLGSYFGASV CAVDLNADGF SDLLVGAPMQ STIREEGRVF VYINSGSGAV MNAMETNLVG |
| 361 | SDKYAARFGE SIVNLGDIDN DGFEDVAIGA PQEDDLQGAI YIYNGRADGI SSTFSQRIEG |
| 421 | LQISKSLSMF GQSISGQIDA DNNGYVDVAV GAFRSDSAVL LRTRPVVIVD ASLSHPESVN |
| 481 | RTKFDCVENG WPSVCIDLTL CFSYKGKEVP GYIVLFYNMS LDVNRKAESP PRFYFSSNGT |
| 541 | SDVITGSIQV SSREANCRTH QAFMRKDVRD ILTPIQIEAA YHLGPHVISK RSTEEFPPLQ |
| 601 | PILQQKKEKD IMKKTINFAR FCAHENCSAD LQVSAKIGFL KPHENKTYLA VGSMKTLMLN |
| 661 | VSLFNAGDDA YETTLHVKLP VGLYFIKILE LEEKQINCEV TDNSGVVQLD CSIGYIYVDH |
| 721 | LSRIDISFLL DVSSLSRAEE DLSITVHATC ENEEEMDNLK HSRVTVAIPL KYEVKLTVHG |
| 781 | FVNPTSFVYG SNDENEPETC MVEKMNLTFH VINTGNSMAP NVSVEIMVPN SFSPQTDKLF |
| 841 | NILDVQTTTG ECHFENYQRV CALEQQKSAM QTLKGIVRFL SKTDKRLLYC IKADPHCLNF |
| 901 | LCNFGKMESG KEASVHIQLE GRPSILEMDE TSALKFEIRA TGFPEPNPRV IELNKDENVA |
| 961 | HVLLEGLHHQ RPKRYFTIVI ISSSLLLGLI VLLLISYVMW KAGFFKRQYK SILQEENRRD |
| 1021 | SWSYINSKSN DD |

Precursor human β7 integrin polypeptide (SEQ ID NO: 13) with the signal sequence underlined and the transmembrane region shown in bold.

| (SEQ ID NO: 13) | |
|---|---|
| 1 | MVALPMVLVL LLVLSRGESE LDAKIPSTGD ATEWRNPHLS MLGSCQPAPS CQKCILSHPS |
| 61 | CAWCKQLNFT ASGEAEARRC ARREELLARG CPLEELEEPR GQQEVLQDQP LSQGARGEGA |
| 121 | TQLAPQRVRV TLRPGEPQQL QVRFLRAEGY PVDLYYLMDL SYSMKDDLER VRQLGHALLV |
| 181 | RLQEVTHSVR IGFGSFVDKT VLPFVSTVPS KLRHPCPTRL ERCQSPFSFH HVLSLTGDAQ |
| 241 | AFEREVGRQS VSGNLDSPEG GFDAILQAAL CQEQIGWRNV SRLLVFTSDD TFHTAGDGKL |
| 301 | GGIFMPSDGH CHLDSNGLYS RSTEFDYPSV GQVAQALSAA NIQPIFAVTS AALPVYQELS |
| 361 | KLIPKSAVGE LSEDSSNVVQ LIMDAYNSLS STVTLEHSSL PPGVHISYES QCEGPEKREG |
| 421 | KAEDRGQCNH VRINQTVTFW VSLQATHCLP EPHLLRLRAL GFSEELIVEL HTLCDCNCSD |
| 481 | TQPQAPHCSD GQGHLQCGVC SCAPGRLGRL CECSVAELSS PDLESGCRAP NGTGPLCSGK |
| 541 | GHCQCGRCSC SGQSSGHLCE CDDASCERHE GILCGGFGRC QCGVCHCHAN RTGRACECSG |
| 601 | DMDSCISPEG GLCSGHGRCK CNRCQCLDGY YGALCDQCPG CKTPCERHRD CAECGAFRTG |
| 661 | PLATNCSTAC AHTNVTLALA PILDDGWCKE RTLDNQLFFF LVEDDARGTV VLRVRPQEKG |
| 721 | ADHTQAIVLG CVGGIVAVGL GLVLAYRLSV EIYDRREYSR FEKEQQQLNW KQDSNPLYKS |
| 781 | AITTTINPRF QEADSPTL |

An exemplary cDNA encoding human α4 integrin is SEQ ID NO: 27. An exemplary cDNA encoding human β7 integrin is SEQ ID NO: 16.

### Soluble α4β1 Agents

Alpha-4, beta-1 integrin is a heterodimer that is composed of α4 integrin and β1 integrin. Both the α4 integrin and the β1 integrin contain a number of domains including a signal sequence, a transmembrane domain, and a cytoplasmic domain. A non-limiting example of a human mature, processed β1 integrin protein is SEQ ID NO: 20. A soluble α4β1 agent is a composition that contains a protein (e.g., a single polypeptide or a heterodimeric protein) that contains an amino acid sequence that is at least 95% (e.g., at least 96%, 97%, 98%, 99% or 100%) identical to a contiguous sequence (e.g., at least 50, 75, 100, 125, 150, 175, 200, 250, 300, 350, 400, 450, 500, 550, 600, 650, 700, 750, 800, or 850 amino acids) in a wild type form of α4 integrin (e.g., precursor or processed α4 integrin, e.g., any one of SEQ ID NOS: 8 and 12) and an amino acid sequence that is at least 95% (e.g., 96%, 97%, 98%, 99%, or 100%) identical to a contiguous sequence (e.g., at least 50, 75, 100, 125, 150, 175, 200, 250, 300, 350, 400, 450, 500, 550, 600, 650, or 700 amino acids) in a wild type form of β1 integrin (e.g., precursor or processed β1 integrin, e.g., any one of SEQ ID NOS: 17 and 20) that is soluble at physiological pH and has the ability to specifically bind to MadCAM-1. In some embodiments, the amino acid sequence that is at least 95% identical to a contiguous sequence in α4 integrin lacks the signal sequence, the transmembrane domain, and cytoplasmic domain of α4 integrin, and/or the amino acid sequence that is at least 95% identical to a contiguous sequence in β1 integrin lacks the signal sequence, the transmembrane domain, and cytoplasmic domain of β1 integrin. In some embodiments, the soluble α4β1 agent contains a polypeptide that contains an additional amino acid sequence (e.g., a sequence that stabilizes the polypeptide or increases the polypeptide's half-life in vivo, e.g., an Fc domain or serum albumin).

In some embodiments, the amino acid sequence that is at least 95% identical to a contiguous sequence in α4 integrin contains a sequence that is at least 95% (e.g., at least 96%, 97%, 98%, 99%, or 100%) identical to a contiguous sequence (e.g., at least 50, 75, 100, 125, 150, 175, 200, 250, 300, 350, 400, 450, 500, 550, 600, 650, 700, 750, 800, or 850 amino acids) present between amino acids 34 and amino acid 977 in SEQ ID NO: 12. In some embodiments, the amino acid sequence that is at least 95% identical to a contiguous sequence in β1 integrin contains a sequence that is at least 95% identical (e.g., at least 96%, 97%, 98%, 99%, or 100% identical) to a contiguous sequence (e.g., at least 50, 75, 100, 125, 150, 175, 200, 250, 300, 350, 400, 450, 500, 550, 600, 650, or 700 amino acids) present between amino acid 21 and amino acid 728 in SEQ ID NO: 17. The mRNA encoding α4 is shown above and the mRNA encoding β1 is shown below. Soluble α4β1 agents can be generated using molecular biology skills known in the art. The ability of a soluble α4β1 agent to bind to MadCAM-1, can be performed using cell binding assays (e.g., cells expressing one or more of MadCAM-1), competitive binding assays with antibodies that specifically bind to MadCAM-1, or surface plasmon resonance (SPR).

Precursor human β1 integrin polypeptide (SEQ ID NO: 17) with the signal sequence underlined and the transmembrane domain shown in bold.

| | |
|---|---|
| 1 | MNLQPIFWIG LISSVCCVFA QTDENRCLKA NAKSCGECIQ AGPNCGWCTN STFLQEGMPT |
| 61 | SARCDDLEAL KKKGCPPDDI ENPRGSKDIK KNKNVTNRSK GTAEKLKPED ITQIQPQQLV |
| 121 | LRLRSGEPQT FTLKFKRAED YPIDLYYLMD LSYSMKDDLE NVKSLGTDLM NEMRRITSDF |
| 181 | RIGFGSFVEK TVMPYISTTP AKLRNPCTSE QNCTSPFSYK NVLSLTNKGE VFNELVGKQR |
| 241 | ISGNLDSPEG GFDAIMQVAV CGSLIGWRNV TRLLVFSTDA GFHFAGDGKL GGIVLPNDGQ |
| 301 | CHLENNMYTM SHYYDYPSIA HLVQKLSENN IQTIFAVTEE FQPVYKELKN LIPKSAVGTL |
| 361 | SANSSNVIQL IIDAYNSLSS EVILENGKLS EGVTISYKSY CKNGVNGTGE NGRKCSNISI |
| 421 | GDEVQFEISI TSNKCPKKDS DSFKIRPLGF TEEVEVILQY ICECECQSEG IPESPKCHEG |
| 481 | NGTFECGACR CNEGRVGRHC ECSTDEVNSE DMDAYCRKEN SSEICSNNGE CVCGQCVCRK |
| 541 | RDNTNEIYSG KFCECDNFNC DRSNGLICGG NGVCKCRVCE CNPNYTGSAC DCSLDTSTCE |
| 601 | ASNGQICNGR GICECGVCKC TDPKFQGQTC EMCQTCLGVC AEHKECVQCR AFNKGEKKDT |
| 661 | CTQECSYFNI TKVESRDKLP QPVQPDPVSH CKEKDVDDCW FYFTYSVNGN NEVMVHVVEN |
| 721 | PECPTGPDII PIVAGVVAGI VLIGLALLLI WKLLMIIHDR REFAKFEKEK MNAKWDTGEN |
| 781 | PIYKSAVTTV VNPKYEGK |

An exemplary cDNA encoding human β1 integrin is SEQ ID NO: 18.

Soluble MadCAM-1 Molecules

The precursor form of MadCAM-1 contains a short cytoplasmic domain, a transmembrane domain, a mucin-like domain, two IgSF domains, and a signal sequence (see, Leung et al., Immunol. Cell. Biol. 74:490-496, 1996; Shyjan et al., J. Immunol. 156:2851-2857, 1996). A soluble MadCAM-1 molecule contains an amino acid sequence that is at least 95% (e.g., at least 96%, 97%, 98%, 99%, or 100%) identical to a contiguous sequence (e.g., at least 50, 75, 100, 125, 150, 175, 200, 225, 250, or 275 amino acids) in a wild type form of MadCAM-1 (e.g., precursor or processed MadCAM-1, e.g., any one of SEQ ID NOS: 1-7 and 11) that is soluble at a physiological pH and has the ability to specifically bind to α4β7 integrin, L-selectin, or α4β1 integrin. In some embodiments, the soluble MadCAM-1 lacks one or more (e.g., two, three, or four of its domains) of its domains. In some embodiments, the soluble MadCAM-1 molecule lacks its signal sequence, its transmembrane domain, and its cytoplasmic domain. In some embodiments, the soluble MadCAM-1 molecule contains an additional amino acid sequence (e.g., a sequence that stabilizes the protein or increases the protein's half-life in vivo, e.g., a Fc domain or serum albumin).

In some embodiments, the soluble MadCAM-1 molecule contains a sequence that is at least 95% identical (e.g., at least 96%, 97%, 98%, 99%, or 100%) to a contiguous sequence (e.g., at least 50, 75, 100, 125, 150, 175, 200, 225, 250, or 275 amino acids) present between amino acid 19 and amino acid 318 in SEQ ID NO: 11 shown below. The mRNA encoding precursor MadCAM-1 is also shown below. Soluble MadCAM-1 molecules can be generated using molecular biology skills known in the art. The ability of a soluble MadCAM-1 molecule to bind to α4β1, α4β7, or L-selectin can be performed using cell binding assays (e.g., cells expressing one or more of α4β1, α4β7, or L-selectin), competitive binding assays with antibodies that specifically bind to one or more of α4β1, α4β7, or L-selectin, or surface plasmon resonance (SPR).

Precursor human MadCAM-1 polypeptide (SEQ ID NO: 11) with the signal sequence underlined and the transmembrane domain shown in bold.

| (SEQ ID NO: 11) | |
|---|---|
| 1 | MDFGLALLLA GLLGLLLGQS LQVKPLQVEP PEPVVAVALG ASRQLTCRLA CADRGASVQW |
| 61 | RGLDTSLGAV QSDTGRSVLT VRNASLSAAG TRVCVGSCGG RTFQHTVQLL VYAFPDQLTV |
| 121 | SPAALVPGDP EVACTAHKVT PVDPNALSFS LLVGGQELEG AQALGPEVQE EEEEPQGDED |
| 181 | VLFRVTERWR LPPLGTPVPP ALYCQATMRL PGLELSHRQA IPVLHSPTSP EPPDTTSPES |
| 241 | PDTTSPESPD TTSQEPPDTT SPEPPDKTSP EPAPQQGSTH TPRSPGSTRT RRPEISQAGP |
| 301 | TQGEVIPTGS SKPAGDQLPA ALWTSSAVLG LLLLALPTYH LWKRCRHLAE DDTHPPASLR |
| 361 | LLPQVSAWAG LRGTGQVGIS PS |

An exemplary cDNA encoding human MadCAM-1 is SEQ ID NO: 14.

### Small Molecule α4β7 Integrin Antagonists

Non-limiting examples of α4β7 integrin antagonists include a number of small molecules. Non-limiting examples of these small molecule α4β7 integrin antagonists include: CT301, thiocarbamates (e.g., U.S. Pat. Nos. 7,015,323 and 7,166,600; each of which is incorporated by reference), sulfasalazines (e.g., U.S. Patent Application Publication No. 2009/0264478; herein incorporated by reference), barbituric acid derivatives (see, e.g., Harriman et al., Bioorg. Med. Chem. Lett. 18:2509-2512, 2008), sulfonamides (e.g., WO 98/53818), TR14035 (see, e.g., Cortijo et al., Br. J. Pharmacol. 147:661-670, 2006), cyclic hexapeptides (e.g., P10 cyclo (Leu-Asp-Thr-Ala-D-Pro-Ala), the peptides described Baer et al., J. Med. Chem. 44:2586-2592, 2001, CWLDVC (TBC 772), and the peptides described in J. Immunol. 158:4180-4186, 1996), tripeptides (see, e.g., the peptides described in Shroff et al., Bioorg. Med. Chem. Lett. 13:1601-1606, 1998), and other small peptides (see, e.g., Schroff et al., Bioorg. Med. Chem. Lett. 6:2495-2500, 1996). Additional examples of small molecule α4β7 integrin antagonists include (2S)-3-(2′,5′-dichlorobiphenyl-4-yl)-2-({[1-(2-methoxybenzoyl)piperidin-3-yl]carbonyl}amino) propanoic acid, BIO5192, AMD 15057, BIO-1211, N-acyl phenylalanine analogues (see, e.g., Chen et al., Bioorg. Med. Chem. Lett. 10:725-727, 2000), and the small molecules described in Mackenzie et al., (Exp. Cell. Res. 276:90-100, 2002), Lobb et al. (Expert. Opin. Invest. Drugs 8:935-945, 1999), Lee et al. (Bioorg. Med. Chem. 17:977-980, 2009), U.S. Patent Application Publication No. 2002/0091142 (incorporated by reference herein), and WO 98/004247. Non-limiting examples of small molecule α4β7 integrin antagonists include the cyclic peptides described in Boer et al. (J. Med. Chem. 44:2586-2592, 2001) (e.g., cyclic hexapeptide P10 cyclo (Leu-Asp-Thr-Ala-D-Pro-Ala), P25 cyclo(Leu-Asp-Thr-Ala-D-Pro-Phe), P28 cyclo(Leu-Asp-Thr-Asp-D-Pro-Phe), P29 cyclo(Leu-Asp-Thr-Asp-D-Pro-His), and P30 cyclo(Leu-Asp-Thr-Asp-D-Pro-Tyr). Additional examples of small molecule α4β7 integrin antagonists are known in the art.

### Small Molecule E-Selectin and L-Selectin Antagonists

In some embodiments, the E-selectin and/or L-selectin antagonist can be a small molecule. Non-limiting examples of small molecules that can act as both an E-selectin antagonist and an L-selectin antagonist include TCB-1269, efomycines (see, e.g., those described in Wienrich et al., J. Invest. Dermatol. 126:882-889, 2006), trihydroxybenzene molecules (see, e.g., those described in Kranich et al., J. Med. Chem. 50:1101-1115, 2007), bimosiamose, and GMI-1070. Additional examples of small molecule L-selectin antagonists and E-selectin antagonists are known in the art.

Methods of Reducing Corneal Inflammation and/or Conjunctival Inflammation

Provided herein are methods of reducing corneal inflammation and/or conjunctival inflammation in a subject that include administering to a subject one or more (e.g., two, three, four, or five) of a MadCAM-1 antagonist, an α4β7 integrin antagonist, a L-selectin antagonist, and an E-selectin antagonist.

In some embodiments, the corneal inflammation is chronic corneal inflammation. In some embodiments, the corneal inflammation is acute corneal inflammation. In some embodiments, the corneal inflammation can be caused by bacterial infection, fungal infection, parasite infection, viral infection (e.g., herpes simplex or herpes zoster), allergy, dry eye disorder, Fuchs' dystrophy, keratoconus, amyloidosis, lattice dystrophy, Stevens Johnson syndrome, physical corneal injury (e.g., abrasion, puncture or trauma, e.g., surgical trauma), Behcet's disease, contact lens wear, corneal graft rejection, dry eye syndrome, or immune keratitis (e.g., peripheral ulcerative keratitis). Additional causes of corneal inflammation are known in the art.

In some embodiments, the conjunctival inflammation is chronic conjunctival inflammation. In some embodiments, the conjunctival inflammation is acute conjunctival inflammation. In some embodiments, the conjunctival inflammation can be caused by viruses, bacteria (e.g., gonorrhea or chlamydia), irritants (e.g., shampoos, dirt, smoke, or chlorine), an allergen, or contact lens wear. Additional causes of conjunctival inflammation are known in the art.

A reduction in corneal inflammation can be detected by the observance or detection a decrease in one or more (e.g., at least two, three, or four) of the following: the number of T-lymphocytes (e.g., effector T-cells) in a cornea, the number of dendritic cells in a cornea, the number of macrophages in a cornea, the number of stimulated monocytes in a cornea, the number of B-cell in a cornea, the number of natural killer cells present in a cornea, the number of eosinophils in a cornea, the number of mast cells in a cornea, the level of redness in a cornea, pain in an eye, irritation, itchiness, burning, and/or dryness of the cornea, swelling around the eye, sensitivity to light, the amount of discharge from an eye, difficulty opening an eyelid, and blurred vision (e.g., as compared to the same subject prior to receiving a treatment (e.g., any of the treatments described herein) or compared to threshold value). In some embodiments, a reduction in corneal inflammation can be assessed by physical examination of the subject (e.g., through the use of microscopic techniques (e.g., in vivo confocal microscopy) or through examination techniques that do not require the use of a microscope). In some embodiments, a reduction in corneal inflammation can be assessed by a medical professional (e.g., a physician, a physician's assistant, a nurse, a nurse's assistant, or a technician).

A reduction in conjunctival inflammation can be detected by the observance or detection a decrease in one or more (e.g., at least two, three, or four) of the following: the number of T-lymphocytes (e.g., effector T-cells) in a conjunctiva, the number of dendritic cells in a conjuctiva, the number of macrophages in a conjunctiva, the number of stimulated monocytes in a conjunctiva, the number of B-cells in a conjunctiva, the number of natural killer cells present in a conjunctiva, the number of eosinophils in a conjunctiva, the number of mast cells in a conjunctiva, the level of redness in the white of an eye or in an eyelid, pain in an eye, irritation, itchiness, burning, and/or dryness of an eye, swelling around the eye, sensitivity to light, the amount of discharge from an eye, difficulty opening an eyelid, and blurred vision (e.g., as compared to the same subject prior to receiving a treatment (e.g., any of the treatments described herein) or compared to threshold value). In some embodiments, a reduction in conjunctival inflammation can be assessed by physical examination of the subject (e.g., through the use of microscopic techniques (e.g., in vivo confocal microscopy) or through examination techniques that do not require the use of a microscope). In some embodiments, a reduction in conjunctival inflammation can be assessed by a medical professional (e.g., a physician, a physician's assistant, a nurse, a nurse's assistant, or a technician).

Some embodiments further include selecting a subject having corneal inflammation and/or conjunctival inflammation (e.g., any type of corneal inflammation and/or conjunctival inflammation described herein or corneal inflammation and/or conjunctival inflammation caused by any of the factors described herein) or a corneal inflammatory disorder and/or a conjunctival inflammatory disorder (e.g., any of the corneal inflammatory disorders and/or conjunctival inflammatory disorders described herein).

In some embodiments, the MadCAM-1 antagonist can be an antibody (e.g., any of the antibodies described herein) or an antigen-binding antibody fragment (e.g., any of the antibody fragments described herein) that specifically binds to MadCAM-1. In some embodiments, the MadCAM-1 antagonist can be a soluble L-selectin molecule (e.g., any of the soluble L-selectin molecules described herein), a soluble α4β7 agent (e.g., any of the soluble α4β7 agents described herein), or a soluble α4β1 agent (e.g., any of the soluble α4β1 agents described herein).

In some embodiments, the α4β7 integrin antagonist can be an antibody (e.g., any of the antibodies described herein) or an antigen-binding antibody fragment (e.g., any of the antibody fragments described herein) that specifically binds to α4β7 integrin, α4 integrin, or integrin. In some embodiments, the α4β7 antagonist is a soluble MadCAM-1 molecule (e.g., any of the soluble MadCAM-1 molecules described herein). In some embodiments, the α4β7 integrin antagonist is a small molecule (a small molecule α4β7 integrin antagonist) (e.g., any of the small molecule α4β7 integrin antagonists described herein or known in the art).

In some embodiments, the L-selectin antagonist can be an antibody (e.g., any of the antibodies described herein) or an antigen-binding antibody fragment (e.g., any of the antibody fragments described herein) that specifically binds to L-selectin. In some embodiments, the L-selectin antagonist is a soluble MadCAM-1 molecule (e.g., any of the soluble MadCAM-1 molecules described herein), a soluble CD34 molecule (e.g., any of the soluble CD34 molecules described herein), a soluble PSGL-1 molecule (e.g., any of the soluble PSGL-1 molecules described herein), or a soluble GlyCAM-1 molecule (e.g., any of the soluble GlyCAM-1 molecules described herein). In some embodiments, the L-selectin antagonist is a small molecule (a small molecule L-selectin antagonist) (e.g., any of the small molecule L-selectin antagonists described herein or known in the art).

In some embodiments, the E-selectin antagonist can be an antibody (e.g., any of the antibodies described herein) or an antigen-binding antibody fragment (e.g., any of the antibody fragments described herein) that specifically binds to E-selectin. In some embodiments, the E-selectin antagonist is a small molecule (a small molecule E-selectin antagonist) (e.g., any of the small molecule E-selectin antagonists described herein or known in the art).

In some embodiments, when two or more agents (e.g., MadCAM-1 antagonist(s), α4β7 integrin antagonist(s), L-selectin antagonist(s), and/or E-selectin antagonist(s)) are administered to the subject, the two or more agents can be any combination of the agents described herein (e.g., a combination of at least one antibody or antigen-binding antibody fragment and at least one soluble selectin-L molecule, soluble MadCAM-1 molecule, soluble α4β7 agent, soluble α4β1 agent, soluble CD34 molecule, soluble PSGL-1 molecule, and/or soluble GlyCAM-1 molecule; a combination of two or more antibodies or antigen-binding antibody fragments; a combination of two or more of a soluble selectin-L molecule, a soluble MadCAM-1 molecule, a soluble α4β7 agent, a soluble α4β1 agent, a soluble CD34 molecule, a soluble PSGL-1 molecule, and a soluble GlyCAM-1 molecule; a combination of one or more antibodies or antigen-binding antibody fragments and one or more small molecule α4β7 integrin antagonists, small molecule L-selectin antagonists, and small molecule E-selectin antagonists; or a combination of two or more small molecule α4β7 integrin antagonists, small molecule L-selectin antagonists, and small molecule E-selectin antagonists). In some embodiments where two or more agents (selected from a MadCAM-1 antagonist, an α4β7 integrin antagonist, a L-selectin antagonist, and an E-selectin antagonist) are administered, the two or more agents can be formulated in a single composition (e.g., any of the compositions described herein). In some embodiments, one or more of a MadCAM-1 antagonist, an α4β7 integrin antagonist, an L-selectin antagonist, and a E-selectin antagonist are formulated for ocular administration (e.g., an eye drop formulation). In some embodiments, one or more of a MadCAM-1 antagonist, an α4β7 integrin antagonist, an L-selectin antagonist, and a E-selectin antagonist are formulated for systemic administration (e.g., oral, intramuscular, intravenous, intaarterial, subcutaneous, or intraperitoneal injection).

In some embodiments, the subject is administered one or more of a MadCAM-1 antagonist, an α4β7 integrin antagonist, a L-selectin antagonist, and a E-selectin antagonist by intravenous, intaarterial, intramuscular, ocular, nasal, subcutaneous, or intraperitoneal administration. The amount of one or more of a MadCAM-1 antagonist, an α4β7 integrin antagonist, a L-selectin antagonist, and an E-selectin antagonist can be the amount (e.g., the amount of one or more agents) that results in a observable or detectable decrease in one or more physical characteristics of corneal inflammation and/or conjunctival inflammation (e.g., any of the physical characteristics of corneal inflammation and/or conjunctival inflammation described herein). A medical professional can determine the appropriate dosage of one or more of a MadCAM-1 antagonist, an α4β7 integrin antagonist, a L-selectin antagonist, and an E-selectin antagonist to administer to the subject based on a number of factors (e.g., the subject's age, general health, sex, and body weight). Exemplary dosages of a MadCAM-1 antagonist, an α4β7 integrin antagonist, a L-selectin antagonist, and an E-selectin antagonist to be administered to the subject are described herein. In some embodiments, the one or more of a MadCAM-1 antagonist, an α4β7 integrin antagonist, a L-selectin antagonist, and an E-selectin antagonist are formulated in a physiologically acceptable excipient or buffer. In some embodiments where two or more agents (agents from the group of a MadCAM-1 antagonist, an α4β7 integrin antagonist, a L-selectin antagonist, and an E-selectin antagonist) are administered to the subject, the agents can, e.g., be administered in separate compositions (e.g., by the same (e.g., ocular administration) or different routes of administration (e.g., any combination of the various routes of administration described herein, e.g., one composition administered by ocular administration and one composition administered by subcutaneous or oral administration)).

In some embodiments, one or more of a MadCAM-1 antagonist, an α4β7 integrin antagonist, a L-selectin antagonist, and an E-selectin antagonist are administered to the subject at least once every two months (e.g., at least once every month, at least once every two weeks, at least once a week, or at least once, twice, or three times a day). In some embodiments, the subject can be periodically administered one or more of a MadCAM-1 antagonist, an α4β7 integrin antagonist, a L-selectin antagonist, and an E-selectin antagonist over a period of at least one week (e.g., at least one month, two months, six months, one year, and two years).

In some embodiments, a subject is administered at least one or both of a MadCAM-1 antagonist (e.g., any of the MadCAM-1 antagonists described herein) and an α4β7 integrin antagonist (e.g., any of the α4β7 integrin antagonists described herein). In some embodiments, a MadCAM-1 antagonist and an α4β7 integrin antagonist are formulated in the same composition (e.g., a composition for oral or ocular administration). In some embodiments, a MadCAM-1 antagonist and an α4β7 integrin antagonist are formulated in separate compositions. In some embodiments, a MadCAM-1 antagonist and an α4β7 integrin antagonist are administered to the subject at least once every two months (e.g., once every month, once every two week, or at least once a day (e.g., twice a day, three times a day, four times a day, or five times a day)).

Some embodiments further include administering to the subject one or more additional agents (e.g., an antibiotic, anti-parasitic agent, an anti-viral agent, an anti-fungal agent, and an anti-inflammatory agent). In some embodiments, the one or more additional agents are present in the same formulation with one or more of a MadCAM-1 antagonist, an α4β7 integrin antagonist, a L-selectin antagonist, and an E-selectin antagonist.

In some embodiments, the subject has previously been diagnosed or identified as having corneal inflammation and/or conjunctival inflammation, or as having a corneal inflammatory disorder and/or a conjunctival inflammatory disorder. In some embodiments, the subject does not present with one or more symptoms of corneal inflammation and/or conjunctival inflammation that can be detected without the use of a microscope. In some embodiments, the subject may be resistant or respond poorly to other forms of treatment for corneal inflammation and/or conjunctival inflammation. In some embodiments, the subject was previously administered another treatment for corneal inflammation and/or conjunctival inflammation, and the subject ceases taking the previously administered treatment prior to being administered one or more of a MadCAM-1 antagonist, an α4β7 integrin antagonist, a L-selectin antagonist, and an E-selectin antagonist. In some embodiments, the subject was previously administered another treatment for corneal inflammation and/or conjunctival inflammation, and the subject is administered the previous treatment in addition to one or more of a MadCAM-1 antagonist, an α4β7 integrin antagonist, a L-selectin antagonist, and an E-selectin antagonist.

In some embodiments, the subject is a child, teenager, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

Methods of Reducing Inflammatory Cell Recruitment to the Cornea and/or Conjunctiva

Provided herein are methods of decreasing inflammatory cell (e.g., any of the inflammatory cells described herein, e.g., dendritic cell) recruitment to the cornea and/or conjunctiva in a subject that include administering to a subject one or more of a MadCAM-1 antagonist, an α4β7 integrin antagonist, a L-selectin antagonist, and an E-selectin antagonist. In some embodiments, the decreased inflammatory cell (e.g., dendritic cell) recruitment is inflammatory cell (e.g., dendritic cell) recruitment to the corneal epithelium. In some embodiments, the decreased inflammatory cell (e.g., dendritic cell) recruitment is inflammatory cell (e.g., dendritic cell) recruitment to the anterior or posterior stroma of the cornea. In some embodiments, the decrease inflammatory cell (e.g., dendritic cell) recruitment is inflammatory cell (e.g., dendritic cell) recruitment to the conjunctiva.

A decrease in inflammatory cell (e.g., dendritic cell) recruitment to the cornea in a subject can be indirectly detected by the observance or detection of a decrease in one or more (e.g., at least two, three, or four) of the following: the number of other immunological cells present in the cornea (e.g., one or more of the number of T-lymphocytes (e.g., T-cell effector cells), macrophages, stimulated monocytes, B-cells, natural killer cells, eosinophils, and mast cells), the level of redness in a cornea, pain in an eye, irritation, itchiness, burning, and/or dryness of the cornea, swelling around the eye, sensitivity to light, the amount of discharge from an eye, difficulty opening eyelid, and blurred vision (e.g., as compared to the same subject prior to receiving a treatment (e.g., any of the treatments described herein), compared to a subject that has corneal inflammation or a corneal inflammatory disease, or compared to threshold value). In some embodiments, a decrease in inflammatory cell (e.g., dendritic cell) recruitment to the cornea in a subject can be directly detected by observance or detection of a decrease in the number of one or more types of inflammatory cells (e.g., dendritic cells) present in the cornea (e.g., the corneal epithelium or the anterior or posterior stroma of the cornea) (e.g., as compared to the same subject prior to receiving a treatment (e.g., any of the treatments described herein) or compared to threshold value). The detection of the presence of different immunological cell types in the cornea of the subject can be determined using a microscopic technique (e.g., in vivo confocal microscopy as described herein and other techniques known in the art). In some embodiments, a decrease in the migration of inflammatory cells (e.g., dendritic cells) to the cornea can be indirectly assessed through examination techniques that do not require the use of a microscope). In some embodiments, a decrease in the migration of inflammatory cells (e.g., dendritic cells) (using any of the methods described herein or known in the art) can be assessed by a medical professional (e.g., a physician, a physician's assistant, a nurse, a nurse's assistant, or a technician).

A decrease in inflammatory cell (e.g., dendritic cell) recruitment to the conjunctiva in a subject can be indirectly detected by the observance or detection of a decrease in one or more (e.g., at least two, three, or four) of the following: the number of other immunological cells present in the conjunctiva (e.g., one or more of the number of T-lymphocytes (e.g., T-cell effector cells), macrophages, stimulated monocytes, B-cells, natural killer cells, eosinophils, and mast cells), the level of redness in the white of an eye or an eyelid, pain in an eye, irritation, itchiness, burning, and/or dryness of an eye, swelling around the eye, sensitivity to light, the amount of discharge from an eye, difficulty opening eyelid, and blurred vision (e.g., as compared to the same subject prior to receiving a treatment (e.g., any of the treatments described herein), compared to a subject that has conjunctival inflammation or a conjunctival inflammatory disease, or compared to threshold value). In some embodiments, a decrease in inflammatory cell (e.g., dendritic cell) recruitment to the conjunctiva in a subject can be directly detected by observance or detection of a decrease in the number of one or more types of inflammatory cells (e.g., dendritic cells) present in the conjunctiva (e.g., as compared to the same subject prior to receiving a treatment (e.g., any of the treatments described herein) or compared to threshold value). The detection of the presence of different immunological cell types in the conjunctiva of the subject can be determined using a microscopic technique (e.g., in vivo confocal microscopy as described herein and other techniques known in the art). In some embodiments, a decrease in the migration of inflammatory cells (e.g., dendritic cells) to the conjunctiva can be indirectly assessed through examination techniques that do not require the use of a microscope). In some embodiments, a decrease in the migration of inflammatory cells (e.g., dendritic cells) (using any of the methods described herein or known in the art) can be assessed by a medical professional (e.g., a physician, a physician's assistant, a nurse, a nurse's assistant, or a technician).

Some embodiments further include selecting a subject having corneal inflammation and/or conjunctival inflammation (e.g., any type of corneal inflammation and/or conjunctival inflammation described herein, or corneal inflammation and/or conjunctival inflammation caused by any of the factors described herein) or a corneal inflammatory disorder and/or a conjunctival inflammatory disorder (e.g., any of the corneal inflammatory disorders and/or conjunctival inflammatory disorders described herein). Some embodiments include selecting a subject suspected of having or presenting with one or more symptoms of corneal inflammation and/or conjunctival inflammation, or with one or more symptoms of a corneal inflammatory disorder and/or a conjunctival inflammatory disorder (e.g., any of the symptoms described herein). Some embodiments include selecting a subject diagnosed as having corneal inflammation and/or conjunctival inflammation, or diagnosed as having a corneal inflammatory disorder and/or a conjunctival inflammatory disorder. Some embodiments include selecting a subject at increased risk of developing corneal and/or conjunctival inflammation, or at increased risk of developing a corneal inflammatory disorder and/or a conjunctival inflammatory disorder.

In some embodiments, the MadCAM-1 antagonist can be an antibody (e.g., any of the antibodies described herein) or an antigen-binding antibody fragment (e.g., any of the antibody fragments described herein) that specifically binds to MadCAM-1. In some embodiments, the MadCAM-1 antagonist can be a soluble L-selectin molecule (e.g., any of the soluble L-selectin molecules described herein), a soluble α4β7 agent (e.g., any of the soluble α4β7 agents described herein), or a soluble α4β1 agent (e.g., any of the soluble α4β1 agents described herein).

In some embodiments, the α4β7 integrin antagonist can be an antibody (e.g., any of the antibodies described herein) or an antigen-binding antibody fragment (e.g., any of the antibody fragments described herein) that specifically binds to α4β7 integrin. In some embodiments, the α4β7 antagonist is a soluble MadCAM-1 molecule (e.g., any of the soluble MadCAM-1 molecules described herein). In some embodiments, the α4β7 integrin antagonist is a small molecule (a small molecule α4β7 integrin antagonist) (e.g., any of the small molecule α4β7 integrin antagonists described herein or known in the art).

In some embodiments, the L-selectin antagonist can be an antibody (e.g., any of the antibodies described herein) or an antigen-binding antibody fragment (e.g., any of the antibody fragments described herein) that specifically binds to L-selectin. In some embodiments, the L-selectin antagonist is a soluble MadCAM-1 molecule (e.g., any of the soluble MadCAM-1 molecules described herein), a soluble CD34 molecule (e.g., any of the soluble CD34 molecules described herein), a soluble PSGL-1 molecule (e.g., any of the soluble PSGL-1 molecules described herein), or a soluble GlyCAM-1 molecule (e.g., any of the soluble GlyCAM-1 molecules described herein). In some embodiments, the L-selectin antagonist is a small molecule (a small molecule L-selectin antagonist) (e.g., any of the small molecule L-selectin antagonists described herein or known in the art).

In some embodiments, the E-selectin antagonist can be an antibody (e.g., any of the antibodies described herein) or an antigen-binding antibody fragment (e.g., any of the antibody fragments described herein) that specifically binds to E-selectin. In some embodiments, the E-selectin antagonist is a small molecule (a small molecule E-selectin antagonist) (e.g., any of the small molecule E-selectin antagonists described herein or known in the art).

In some embodiments, when two or more agents (e.g., MadCAM-1 antagonist(s), α4β7 integrin antagonist(s), L-selectin antagonist(s), and/or E-selectin antagonist(s)) are administered to the subject, the two or more agents can be any combination of the agents described herein (e.g., a combination of at least one antibody or antigen-binding antibody fragment and at least one soluble selectin-L molecule, soluble MadCAM-1 molecule, soluble α4β7 agent, soluble α4β1 agent, soluble CD34 molecule, soluble PSGL-1 molecule, and/or soluble GlyCAM-1 molecule; a combination of two or more antibodies or antigen-binding antibody fragments; a combination of two or more of a soluble selectin-L molecule, a soluble MadCAM-1 molecule, a soluble α4β7 agent, a soluble α4β1 agent, a soluble CD34 molecule, a soluble PSGL-1 molecule, and a soluble GlyCAM-1 molecule; a combination of one or more antibodies or antigen-binding antibody fragments and one or more small molecule α4β7 integrin antagonists, small molecule L-selectin antagonists, and small molecule E-selectin antagonists; or a combination of two or more small molecule α4β7 integrin antagonists, small molecule L-selectin antagonists, and small molecule E-selectin antagonists). In some embodiments where two or more agents (selected from a MadCAM-1 antagonist, an α4β7 integrin antagonist, a L-selectin antagonist, and an E-selectin antagonist) are administered, the two or more agents can be formulated in a single composition (e.g., any of the compositions described herein). In some embodiments, one or more of a MadCAM-1 antagonist, an α4β7 integrin antagonist, an L-selectin antagonist, and a E-selectin antagonist are formulated for ocular administration (e.g., an eye drop formulation). In some embodiments, one or more of a MadCAM-1 antagonist, an α4β7 integrin antagonist, an L-selectin antagonist, and a E-selectin antagonist are formulated for systemic administration (e.g., oral, intramuscular, intravenous, intaarterial, subcutaneous, or intraperitoneal injection).

In some embodiments, the subject is administered one or more of a MadCAM-1 antagonist, an α4β7 integrin antagonist, a L-selectin antagonist, and a E-selectin antagonist by intravenous, intaarterial, intramuscular, ocular, nasal, subcutaneous, or intraperitoneal administration. The amount of one or more of a MadCAM-1 antagonist, an α4β7 integrin antagonist, a L-selectin antagonist, and an E-selectin antagonist can be the amount (e.g., the amount of one or more agents) that results in a observable or detectable decrease in inflammatory cell (e.g., dendritic cell) recruitment to the cornea and/or conjunctiva, and/or decreases one or more physical characteristics of corneal inflammation and/or conjunctival inflammation (e.g., any of the physical characteristics of corneal inflammation and/or conjunctival inflammation described herein). A medical professional can determine the appropriate dosage of one or more of a MadCAM-1 antagonist, an α4β7 integrin antagonist, a L-selectin antagonist, and an E-selectin antagonist to administer to the subject based on a number of factors (e.g., the subject's age, general health, sex, and body weight). Exemplary dosages of a MadCAM-1 antagonist, an α4β7 integrin antagonist, a L-selectin antagonist, and an E-selectin antagonist to be administered to the subject are described herein. In some embodiments, the one or more of a MadCAM-1 antagonist, an α4β7 integrin antagonist, a L-selectin antagonist, and an E-selectin antagonist are formulated in a physiologically acceptable excipient or buffer. In some embodiments where two or more agents (agents from the group of a MadCAM-1 antagonist, an α4β7 integrin antagonist, a L-selectin antagonist, and an E-selectin antagonist) are administered to the subject, the agents can, e.g., be administered in separate compositions (e.g., by the same (e.g., ocular administration) or different routes of administration (e.g., any combination of the various routes of administration described herein, e.g., one composition administered by ocular administration and one composition administered by subcutaneous or oral administration)).

In some embodiments, one or more of a MadCAM-1 antagonist, an α4β7 integrin antagonist, a L-selectin antagonist, and an E-selectin antagonist are administered to the subject at least once every two months (e.g., at least once every month, at least once every two weeks, at least once a week, or at least once, twice, or three times a day). In some embodiments, the subject can be periodically administered one or more of a MadCAM-1 antagonist, an α4β7 integrin antagonist, a L-selectin antagonist, and an E-selectin antagonist over a period of at least one week (e.g., at least one month, two months, six months, one year, and two years).

In some embodiments, a subject is administered at least one or both of a MadCAM-1 antagonist (e.g., any of the MadCAM-1 antagonists described herein) and an α4β7 integrin antagonist (e.g., any of the α4β7 integrin antagonists described herein). In some embodiments, a MadCAM-1 antagonist and an α4β7 integrin antagonist are formulated in the same composition (e.g., a composition for oral or ocular administration). In some embodiments, a MadCAM-1 antagonist and an α4β7 integrin antagonist are formulated in separate compositions. In some embodiments, a MadCAM-1 antagonist and an α4β7 integrin antagonist are administered to the subject at least once every two months (e.g., once every month, once every two week, or at least once a day (e.g., twice a day, three times a day, four times a day, or five times a day)).

Some embodiments further include administering to the subject one or more additional agents (e.g., an antibiotic, anti-parasitic agent, an anti-viral agent, an anti-fungal agent, and an anti-inflammatory agent). In some embodiments, the one or more additional agents are present in the same formulation with one or more of a MadCAM-1 antagonist, an α4β7 integrin antagonist, a L-selectin antagonist, and an E-selectin antagonist. In some embodiments, the subject has previously been diagnosed or identified as having corneal inflammation and/or conjunctival inflammation, or as having a corneal inflammatory disorder and/or a conjunctival inflammatory disorder. In some embodiments, the subject is at increased risk of developing corneal and/or conjunctival inflammation, or is at increased risk of developing a corneal inflammatory disorder and/or a conjunctival inflammatory disorder. In some embodiments, the subject is suspected of having or presents with one or more symptoms of corneal inflammation and/or conjunctival inflammation, or is suspected of having or presents with one or more symptoms of a corneal inflammatory disorder and/or a conjunctival inflammatory disorder. In some embodiments, the subject does not present with one or more symptoms of corneal inflammation and/or conjunctival inflammation that can be detected without the use of a microscope. In some embodiments, the subject may be resistant or respond poorly to other forms of treatment for corneal inflammation and/or conjunctival inflammation. In some embodiments, the subject was previously administered another treatment for corneal inflammation and/or conjunctival inflammation, and the subject ceases taking the previously administered treatment prior to being administered one or more of a MadCAM-lantagonist, an α4β7 integrin antagonist, a L-selectin antagonist, and an E-selectin antagonist. In some embodiments, the subject was previously administered another treatment for corneal inflammation and/or conjunctival inflammation, and the subject is administered the previous treatment in addition to one or more of a MadCAM-1 antagonist, an α4β7 integrin antagonist, a L-selectin antagonist, and an E-selectin antagonist.

In some embodiments, the subject is a child, teenager, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

Methods of Treating Corneal Inflammatory Disorders and/or Conjunctival Inflammatory Disorders

Provided herein are methods of treating a corneal inflammatory disorder (e.g., any of the corneal inflammatory disorders described herein or known in the art) and/or a conjunctival inflammatory disorder (e.g., any of the conjuctival inflammatory disorders described herein or known in the art) in a subject that include administering to a subject one or more of a MadCAM-1 antagonist, an α4β7 integrin antagonist, and L-selectin antagonist, and an E-selectin antagonist.

Successful treatment of a corneal inflammatory disorder in a subject can be detected by the observance or detection of a decrease in one or more (e.g., at least two, three, or four) of the following: the number of T-lymphocytes (e.g., effector T-cells) in a cornea, the number of dendritic cells in a cornea, the number of macrophages in a cornea, the number of stimulated monocytes in a cornea, the number of B-cells in a cornea, the number of natural killer cells in a cornea, the number of eosinophils in a cornea, the number of mast cells in a cornea, the level of redness in a cornea, pain in an eye, irritation, itchiness, burning, and/or dryness of the cornea, swelling around the eye, sensitivity to light, the amount of discharge from an eye, difficulty opening an eyelid, and blurred vision (e.g., as compared to the same subject prior to receiving a treatment (e.g., any of the treatments described herein), compared to a subject having corneal inflammation or a corneal inflammatory disease, or compared to threshold value). In some embodiments, a treatment of a corneal inflammatory disorder can be assessed by physical examination of the subject (e.g., through the use of microscopic techniques (e.g., in vivo confocal microscopy) or through examination techniques that do not require the use of a microscope). In some embodiments, successful treatment of a corneal inflammatory disorder can be assessed by a medical professional (e.g., a physician, a physician's assistant, a nurse, a nurse's assistant, or a technician).

Successful treatment of a conjuctival inflammatory disorder in a subject can be detected by the observance or detection of a decrease in one or more (e.g., at least two, three, or four) of the following: the number of T-lymphocytes (e.g., effector T-cells) in a conjunctiva, the number of dendritic cells in a conjunctiva, the number of macrophages in a conjunctiva, the number of stimulated monocytes in a conjunctiva, the number of B-cells in a conjunctiva, the number of natural killer cells in a conjunctiva, the number of eosinophils in a conjunctiva, the number of mast cells in a conjunctiva, the level of redness in a white of an eye or an eyelid, pain in an eye, irritation, itchiness, burning, and/or dryness of an eye, swelling around the eye, sensitivity to light, the amount of discharge from an eye, difficulty opening an eyelid, and blurred vision (e.g., as compared to the same subject prior to receiving a treatment (e.g., any of the treatments described herein), compared to a subject having conjunctival inflammation or a conjunctival inflammatory disease, or compared to threshold value). In some embodiments, a treatment of a conjunctival inflammatory disorder can be assessed by physical examination of the subject (e.g., through the use of microscopic techniques (e.g., in vivo confocal microscopy) or through examination techniques that do not require the use of a microscope). In some embodiments, successful treatment of a conjunctival inflammatory disorder can be assessed by a medical professional (e.g., a physician, a physician's assistant, a nurse, a nurse's assistant, or a technician).

Some embodiments further include selecting a subject having corneal inflammation and/or conjunctival inflammation (e.g., any type of corneal inflammation and/or conjunctival inflammation described herein, or corneal inflammation and/or conjunctival inflammation caused by any of the factors described herein) or a corneal inflammatory disorder and/or a conjunctival inflammatory disorder (e.g., any of the corneal inflammatory disorders and/or conjunctival inflammatory disorders described herein). Some embodiments include selecting a subject suspected of having or presenting with one or more symptoms of corneal inflammation and/or conjunctival inflammation, or with one or more symptoms of a corneal inflammatory disorder and/or a conjunctival inflammatory disorder (e.g., any of the symptoms described herein). Some embodiments include selecting a subject diagnosed as having corneal inflammation and/or conjunctival inflammation, or diagnosed as having a corneal inflammatory disorder and/or a conjunctival inflammatory disorder. Some embodiments include selecting a subject at increased risk of developing corneal and/or conjunctival inflammation, or at increased risk of developing a corneal inflammatory disorder and/or a conjunctival inflammatory disorder.

In some embodiments, the MadCAM-1 antagonist can be an antibody (e.g., any of the antibodies described herein) or an antigen-binding fragment (e.g., any of the antigen-binding antibody fragments described herein) that specifically binds to MadCAM-1. In some embodiments, the MadCAM-1 antagonist can be a soluble L-selectin molecule (e.g., any of the soluble L-selectin molecules described herein), a soluble α4β7 agent (e.g., any of the soluble α4β7 agents described herein), or a soluble α4β1 agent (e.g., any of the soluble α4β1 agents described herein).

In some embodiments, the α4β7 integrin antagonist can be an antibody (e.g., any of the antibodies described herein) or an antigen-binding antibody fragment (e.g., any of the antibody fragments described herein) that specifically binds to α4β7 integrin. In some embodiments, the α4β7 antagonist is a soluble MadCAM-1 molecule (e.g., any of the soluble MadCAM-1 molecules described herein). In some embodiments, the α4β7 integrin antagonist is a small molecule (a small molecule α4β7 integrin antagonist) (e.g., any of the small molecule α4β7 integrin antagonists described herein or known in the art). In some embodiments, the L-selectin antagonist can be an antibody (e.g., any of the antibodies described herein) or an antigen-binding antibody fragment (e.g., any of the antibody fragments described herein) that specifically binds to L-selectin. In some embodiments, the L-selectin antagonist is a soluble MadCAM-1 molecule (e.g., any of the soluble MadCAM-1 molecules described herein), a soluble CD34 molecule (e.g., any of the soluble CD34 molecules described herein), a soluble PSGL-1 molecule (e.g., any of the soluble PSGL-1 molecules described herein), or a soluble GlyCAM-1 molecule (e.g., any of the soluble GlyCAM-1 molecules described herein). In some embodiments, the L-selectin antagonist is a small molecule (a small molecule L-selectin antagonist) (e.g., any of the small molecule L-selectin antagonists described herein or known in the art).

In some embodiments, the E-selectin antagonist can be an antibody (e.g., any of the antibodies described herein) or an antigen-binding antibody fragment (e.g., any of the antibody fragments described herein) that specifically binds to E-selectin. In some embodiments, the E-selectin antagonist is a small molecule (a small molecule E-selectin antagonist) (e.g., any of the small molecule E-selectin antagonists described herein or known in the art).

In some embodiments, when two or more agents (e.g., MadCAM-1 antagonist(s), α4β7 integrin antagonist(s), L-selectin antagonist(s), and/or E-selectin antagonist(s)) are administered to the subject, the two or more agents can be any combination of the agents described herein (e.g., a combination of at least one antibody or antigen-binding antibody fragment and at least one soluble selectin-L molecule, soluble MadCAM-1 molecule, soluble α4β7 agent, soluble α4β1 agent, soluble CD34 molecule, soluble PSGL-1 molecule, and/or soluble GlyCAM-1 molecule; a combination of two or more antibodies or antigen-binding antibody fragments; a combination of two or more of a soluble selectin-L molecule, a soluble MadCAM-1 molecule, a soluble α4β7 agent, a soluble α4β1 agent, a soluble CD34 molecule, a soluble PSGL-1 molecule, and a soluble GlyCAM-1 molecule; a combination of one or more antibodies or antigen-binding antibody fragments and one or more small molecule α4β7 integrin antagonists, small molecule L-selectin antagonists, and small molecule E-selectin antagonists; or a combination of two or more small molecule α4β7 integrin antagonists, small molecule L-selectin antagonists, and small molecule E-selectin antagonists). In some embodiments where two or more agents (selected from a MadCAM-1 antagonist, an α4β7 integrin antagonist, a L-selectin antagonist, and an E-selectin antagonist) are administered, the two or more agents can be formulated in a single composition (e.g., any of the compositions described herein). In some embodiments, one or more of a MadCAM-1 antagonist, an α4β7 integrin antagonist, an L-selectin antagonist, and a E-selectin antagonist are formulated for ocular administration (e.g., an eye drop formulation). In some embodiments, one or more of a MadCAM-1 antagonist, an α4β7 integrin antagonist, an L-selectin antagonist, and a E-selectin antagonist are formulated for systemic administration (e.g., oral, intramuscular, intravenous, intaarterial, subcutaneous, or intraperitoneal injection).

In some embodiments, the subject is administered one or more of a MadCAM-1 antagonist, an α4β7 integrin antagonist, a L-selectin antagonist, and a E-selectin antagonist by intravenous, intaarterial, intramuscular, ocular, nasal, subcutaneous, or intraperitoneal administration. The amount of one or more of a MadCAM-1 antagonist, an α4β7 integrin antagonist, a L-selectin antagonist, and an E-selectin antagonist can be the amount (e.g., the amount of one or more agents) that results in a observable or detectable decrease in one or more physical characteristics of corneal inflammation and/or conjunctival inflammation (e.g., any of the physical characteristics of corneal inflammation and/or conjunctival inflammation described herein). A medical professional can determine the appropriate dosage of one or more of a MadCAM-1 antagonist, an α4β7 integrin antagonist, a L-selectin antagonist, and an E-selectin antagonist to administer to the subject based on a number of factors (e.g., the subject's age, general health, sex, and body weight). Exemplary dosages of a MadCAM-1 antagonist, an α4β7 integrin antagonist, a L-selectin antagonist, and an E-selectin antagonist to be administered to the subject are described herein. In some embodiments, the one or more of a MadCAM-1 antagonist, an α4β7 integrin antagonist, a L-selectin antagonist, and an E-selectin antagonist are formulated in a physiologically acceptable excipient or buffer. In some embodiments where two or more agents (agents from the group of a MadCAM-1 antagonist, an α4β7 integrin antagonist, a L-selectin antagonist, and an E-selectin antagonist) are administered to the subject, the agents can, e.g., be administered in separate compositions (e.g., by the same (e.g., ocular administration) or different routes of administration (e.g., any combination of the various routes of administration described herein, e.g., one composition administered by ocular administration and one composition administered by subcutaneous or oral administration)).

In some embodiments, one or more of a MadCAM-1 antagonist, an α4β7 integrin antagonist, a L-selectin antagonist, and an E-selectin antagonist are administered to the subject at least once every two months (e.g., at least once every month, at least once every two weeks, at least once a week, or at least once, twice, or three times a day). In some embodiments, the subject can be periodically administered one or more of a MadCAM-1 antagonist, an α4β7 integrin antagonist, a L-selectin antagonist, and an E-selectin antagonist over a period of at least one week (e.g., at least one month, two months, six months, one year, and two years).

In some embodiments, a subject is administered at least one or both of a MadCAM-1 antagonist (e.g., any of the MadCAM-1 antagonists described herein) and an α4β7 integrin antagonist (e.g., any of the α4β7 integrin antagonists described herein). In some embodiments, a MadCAM-1 antagonist and an α4β7 integrin antagonist are formulated in the same composition (e.g., a composition for oral or ocular administration). In some embodiments, a MadCAM-1 antagonist and an α4β7 integrin antagonist are formulated in separate compositions. In some embodiments, a MadCAM-1 antagonist and an α4β7 integrin antagonist are administered to the subject at least once every two months (e.g., once every month, once every two week, or at least once a day (e.g., twice a day, three times a day, four times a day, or five times a day)).

Some embodiments further include administering to the subject one or more additional agents (e.g., an antibiotic, anti-parasitic agent, an anti-viral agent, an anti-fungal agent, and an anti-inflammatory agent). In some embodiments, the one or more additional agents are present in the same formulation with one or more of a MadCAM-1 antagonist, an α4β7 integrin antagonist, a L-selectin antagonist, and an E-selectin antagonist.

In some embodiments, the subject has previously been diagnosed or identified as having corneal inflammation and/or conjunctival inflammation, or as having a corneal inflammatory disorder and/or a conjunctival inflammatory disorder. In some embodiments, the subject is at increased risk of developing corneal and/or conjunctival inflammation, or is at increased risk of developing a corneal inflammatory disorder and/or a conjunctival inflammatory disorder. In some embodiments, the subject is suspected of having or presents with one or more symptoms of corneal inflammation and/or conjunctival inflammation, or is suspected of having or presents with one or more symptoms of a corneal inflammatory disorder and/or a conjunctival inflammatory disorder. In some embodiments, the subject does not present with one or more symptoms of corneal inflammation and/or conjunctival inflammation that can be detected without the use of a microscope. In some embodiments, the subject may be resistant or respond poorly to other forms of treatment for corneal inflammation and/or conjunctival inflammation. In some embodiments, the subject was previously administered another treatment for corneal inflammation and/or conjunctival inflammation, and the subject ceases taking the previously administered treatment prior to being administered one or more of a MadCAM-1 antagonist, an α4β7 integrin antagonist, a L-selectin antagonist, and an E-selectin antagonist. In some embodiments, the subject was previously administered another treatment for corneal inflammation and/or conjunctival inflammation, and the subject is administered the previous treatment in addition to one or more of a MadCAM-1 antagonist, an α4β7 integrin antagonist, a L-selectin antagonist, and an E-selectin antagonist.

In some embodiments, the subject is a child, teenager, or an adult (e.g., at least 18, 20, 25, 30, 35, 40, 45, 50, 55, 60, 65, 70, 75, 80, 85, or 90 years old). In some embodiments, the subject is a female. In some embodiments, the subject is a male.

### Compositions

Also provided are compositions containing one or more (e.g., at least two, three, four, or five) MadCAM-1 antagonist(s) (e.g., any of the MadCAM-1 antagonists described herein or known in the art), α4β7 integrin antagonist(s) (e.g., any of the α4β7 integrin antagonists described herein or known in the art), L-selectin antagonist(s) (e.g., any of the L-selectin antagonists described herein or known in the art), and E-selectin antagonist(s) (e.g., any of the E-selectin antagonists described herein or known in the art). In some embodiments, the compositions contain one or more pharmaceutically acceptable excipients or buffers. In some embodiments, the compositions are formulated as a liquid. In some embodiments, the compositions are formulated for ocular administration (e.g., eye drop formulations). In some embodiments, the compositions are formulated for systemic administration (e.g., for oral, intravenous, intraarterial, intramuscular, intraperitoneal, or subcutaneous administration). The methods described herein can include administering these compositions.

Also provided are compositions containing one or more (e.g., at least two, three, four, or five) MadCAM-1 antagonist(s) (e.g., any of the MadCAM-1 antagonists described herein or known in the art), and/or one or more (e.g., at least two, three, or four) α4β7 integrin antagonist(s) (e.g., any of the α4β7 integrin antagonists described here in or known in the art). In some embodiments, the compositions contain one or more pharmaceutically acceptable excipients or buffers. In some embodiments, the compositions are formulated as a liquid. In some embodiments, the compositions are formulated for ocular administration (e.g., eye drop formulations). In some embodiments, the compositions are formulated as a solid (e.g., a pill or capsule). In some embodiments, the compositions are formulated as a slow-release formulation.

Pharmaceutical compositions are formulated to be compatible with their intended route of administration, whether ocular or parenteral (e.g., intravenous, intradermal, subcutaneous, transmucosal (e.g., nasal sprays are formulated for inhalation), or transdermal (e.g., topical ointments, salves, gels, patches or creams as generally known in the art). The compositions can include a sterile diluent (e.g., sterile water or saline), a fixed oil, polyethylene glycol, glycerine, propylene glycol or other synthetic solvents; antibacterial or antifungal agents such as benzyl alcohol or methyl parabens, chlorobutanol, phenol, ascorbic acid, thimerosal, and the like; antioxidants such as ascorbic acid or sodium bisulfate; chelating agents such as ethylenediaminetetraacetic acid; buffers such as acetates, citrates or phosphates; and isotonic agents such as sugars (e.g., dextrose), polyalcohols (e.g., manitol or sorbitol), or salts (e.g., sodium chloride). Liposomal suspensions can also be used as pharmaceutically acceptable carriers (see, e.g., U.S. Pat. No. 4,522,811). Preparations of the compositions can be formulated and enclosed in ampules, disposable syringes or multiple dose vials. Where required (as in, for example, injectable formulations), proper fluidity can be maintained by, for example, the use of a coating such as lecithin, or a surfactant. Absorption of the active ingredient can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Where oral administration is intended, the agent can be included in pills, capsules, troches and the like and can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

The compositions described herein can be formulated for ocular or parenteral administration in dosage unit form (i.e., physically discrete units containing a predetermined quantity of active compound for ease of administration and uniformity of dosage). Toxicity and therapeutic efficacy of compounds can be determined by standard pharmaceutical procedures in cell cultures or experimental animals. One can, for example, determine the LD50 (the dose lethal to 50% of the population) and the ED50 (the dose therapeutically effective in 50% of the population), the therapeutic index being the ratio of LD50:ED50. Agents that exhibit high therapeutic indices are preferred. Where an agent exhibits an undesirable side effect, care should be taken to target that agent to the site of the affected tissue (the aim being to minimize potential damage to unaffected cells and, thereby, reduce side effects). Toxicity and therapeutic efficacy can be determined by other standard pharmaceutical procedures.

In some embodiments, the amount of a MadCAM-1 antagonist present (or each MadCAM-1 antagonist when more than one MadCAM-1 antagonist is present) in a single dose of the composition is between 1 mg and 50 mg, 5 mg and 100 mg, 10 mg to 100 mg, 20 mg to 50 mg, 50 mg to 100 mg, 75 mg to 150 mg, 100 mg to 200 mg, 150 mg to 250 mg. In some embodiments, the amount of a α4β7 integrin antagonist present (or each α4β7 integrin antagonist when more than one α4β7 integrin antagonist is present) in a single dose of the composition is between 1 mg and 50 mg, 5 mg and 100 mg, 10 mg to 100 mg, 20 mg to 50 mg, 50 mg to 100 mg, 75 mg to 150 mg, 100 mg to 200 mg, 150 mg to 250 mg. In some embodiments, the amount of a L-selectin antagonist present (or each L-selectin antagonist when more than one L-selectin antagonist is present) in a single dose of the composition is between 1 mg and 50 mg, 5 mg and 100 mg, 10 mg to 100 mg, 20 mg to 50 mg, 50 mg to 100 mg, 75 mg to 150 mg, 100 mg to 200 mg, 150 mg to 250 mg. In some embodiments, the amount of an E-selectin antagonist present (or each E-selectin antagonist when more than one E-selectin antagonist is present) in a single dose of the composition is between 1 mg and 50 mg, 5 mg and 100 mg, 10 mg to 100 mg, 20 mg to 50 mg, 50 mg to 100 mg, 75 mg to 150 mg, 100 mg to 200 mg, 150 mg to 250 mg.

Some embodiments of the compositions further include one or more additional agents (e.g., one or more (e.g., two, three, four, or five), e.g., agents selected from the group of antibiotics, anti-parasitic agents, an anti-viral agents, an anti-fungal agents, and an anti-inflammatory agents.

### Kits

Also provided herein are kits that contain any of the compositions described herein. In some embodiments, the kits can further include an item for use in administering a composition (e.g., any of the compositions described herein) to the subject (e.g., a syringe, e.g., a pre-filled syringe). In some embodiments, the kits contain one or more doses (e.g., at least two, three, four, five, or six doses) of any of the compositions described herein. In some embodiments, the kit further contains instructions for administering the composition (or a dose of the composition) to a subject having corneal inflammation and/or conjunctival inflammation, or having a corneal inflammatory disorder and/or conjunctival disorder. In some embodiments, the kit further contains instructions for performing any of the methods described herein (e.g., any of the methods of decreasing corneal inflammation and/or conjunctival inflammation, any of the methods of decreasing inflammatory cell (e.g., dendritic cell) migration to the cornea and/or conjunctiva, and any of the methods of treating a corneal inflammatory disorder and/or conjunctival inflammatory disorder described herein). The invention is further described in the following example, which does not limit the scope of the invention described in the claims.

### EXAMPLE

### Example 1. In Vivo Studies of Dendritic Cell Recruitment to the Cornea

A set of experiments was performed in a mouse eye inflammation model to study the role of various adhesion molecules in the rolling and adhesion behavior of dendritic cells at the limbal vessel at the cornea.

Materials and Methods

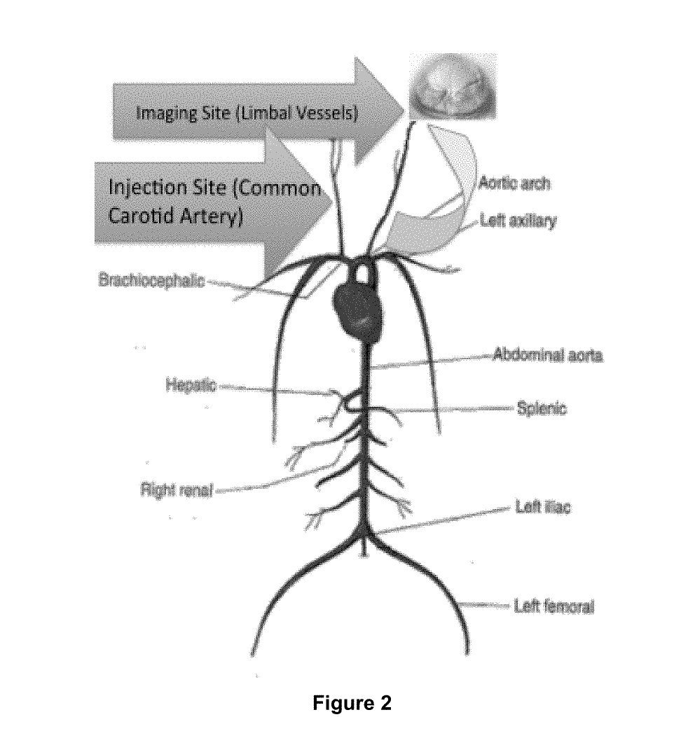

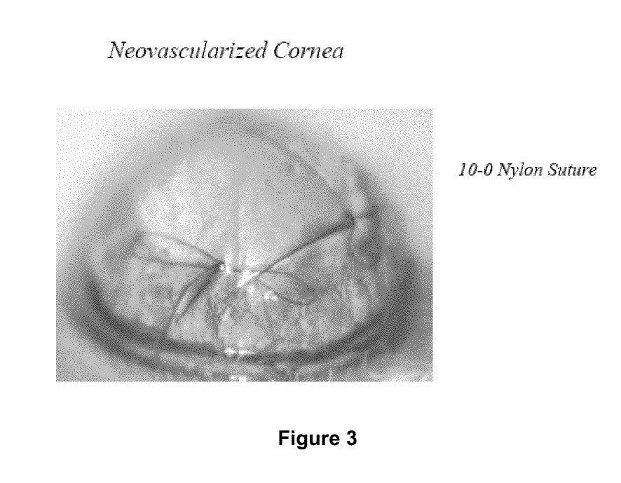

In these experiments, corneal bone marrow-derived dendritic cells (cDC) were purified from Flt-3 melanoma treated mice using L-selectin columns (Miltenyi, Boston, Mass.) and labeled with calcein. Balb/c mice were either left untreated (steady state) or corneal inflammation was induced by suturing one of the eyes of the mouse (suture-induced corneal inflammation) (see, e.g., FIG. 3). One week later calcein-labeled dDC cells (2×10⁶ cells) were injected retrograde through the carotid artery (via catheter) in small boluses of 20-50 μL into the mice (with or without one or more the antibodies described below), and visualized in the blood stream of the contralateral corneal and limbal vasculature using epifluorescent intravital microscopy (EF-IVM; IVM, 500 Mikron Instruments) (n=5 per group) (FIG. 2). (EF-IVM is described in Mempel et al., Curr. Opin. Immunol. 39:2925-2935, 2009.) Specifically, a low-lag silicon-intensified target camera (VE1000SIT; Dage MTI) for ulta-low light real-time video recordings was used with a video-triggered stroboscope system (Chadwick Helmuth), and a time base generator (For-A, Co., Ltd.). Video analysis was carried out at a reduced speed. Rolling (cells that interacted visibly with the endothelium and traveled at a slower velocity than the main blood stream) and non-interacting cells were counted in each microvessel section and averaged. The rolling fraction (percentage of rolling calcein-labeled dendritic cells in the limbal vessel compared to the total number of passing calcein-labeled dendritic cells in the limbal vessel) and sticking efficiency (percentage of calcein-labeled dendritic cells that stick to the limbal vessel wall compared to the total number of passing calcein-labeled dendritic cells) were measured.

The effect of different adhesion molecules on dendritic cell recruitment was determined by intravenous injection with 100 microliters of antibodies that specifically bind to P-selectin, E-selectin, L-selectin, ICAM-1, VCAM-1, or MadCAM-1 at the time the calcein-labelled cells were injected (thirty minutes before intravital microscopy recordings were taken). In the case of L-selectin, the labeled dendritic cells were treated with 10 μl of 0.5 mg/ml anti-selectin antibody for 30 minutes at 4 degrees, and then excess antibody was washed away with PBS prior to injection through the carotid artery. Pertussis toxin (PTX) treatment of the labeled dendritic cells was performed by incubating 0.8 ng of PTX with 20 million labeled dendritic cells for 2 hours at 37° C. Corneal inflammation by suturing was initiated 7 days prior to imaging and or treatment. Homing (recruitment) of the calcein-labeled dendritic cells to normal or inflamed corneas was also studied by ex vivo confocal microscopy (Olympus Fluoview 1000), twenty-four hours after intravenous injection of fluorescently-labeled dendritic cells (with or without antibodies that specifically bind to P-selectin, E-selectin, L-selectin, ICAM-1, VCAM-1, or MadCAM-1).

Results

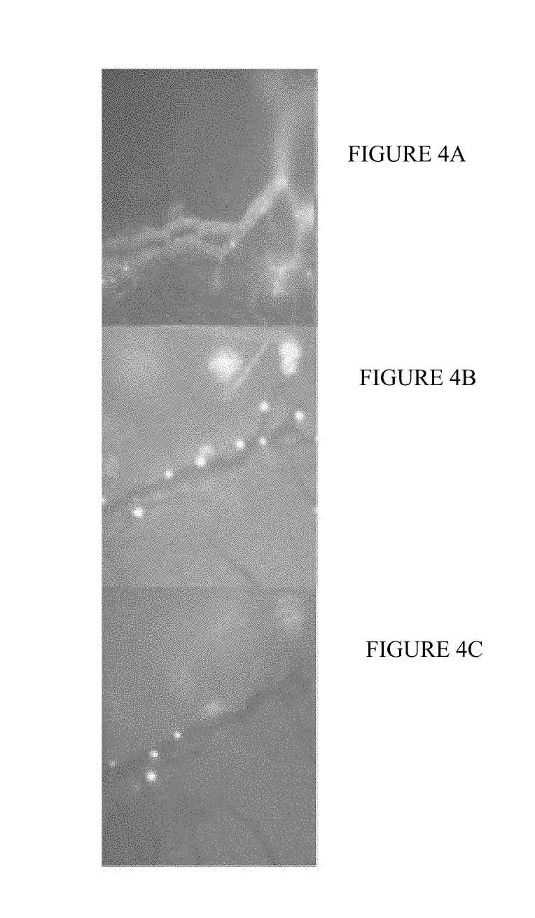

An initial set of experiments show that the injected calcein-labeled dendritic cells are recruited to the limbal vessel in mice that are untreated and mice with suture-induced eye inflammation (both before and after treatment with an anti-VCAM-1 antibody) (see, FIGS. 4A-C).

An additional set of experiments was performed to determine the role of different adhesion molecules in dendritic cell rolling in the limbal vessel of the cornea in both untreated (steady state) mice and in mice with suture-induced inflammation. In these experiments, the mice were left untreated or corneal inflammation was induced (suture-induced inflammation), a week later the mice were administered calcein-labelled dendritic cells with or without anti-P-selectin antibodies, anti-L-selectin antibodies, anti-E-selectin antibodies, an anti-PSGL-1 antibodies, a combination of anti-P-selectin and anti-L-selectin antibodies, anti-CD44 antibodies, anti-VCAM-1 antibodies, or anti-MadCAM-1 antibodies, and intravital images were obtained thirty minutes later. In the case of anti-L-selectin antibody treatment, the labeled dendritic cells were treated with the antibody for 30 minutes at 4° C., and washed with phosphate buffered saline prior to injection. Antibody treatment was performed 30 minutes prior to imaging and seven days after suture induced inflammation) in the in vitro confocal microscopy experiments, and 24 hours in advance of the homing experiments.

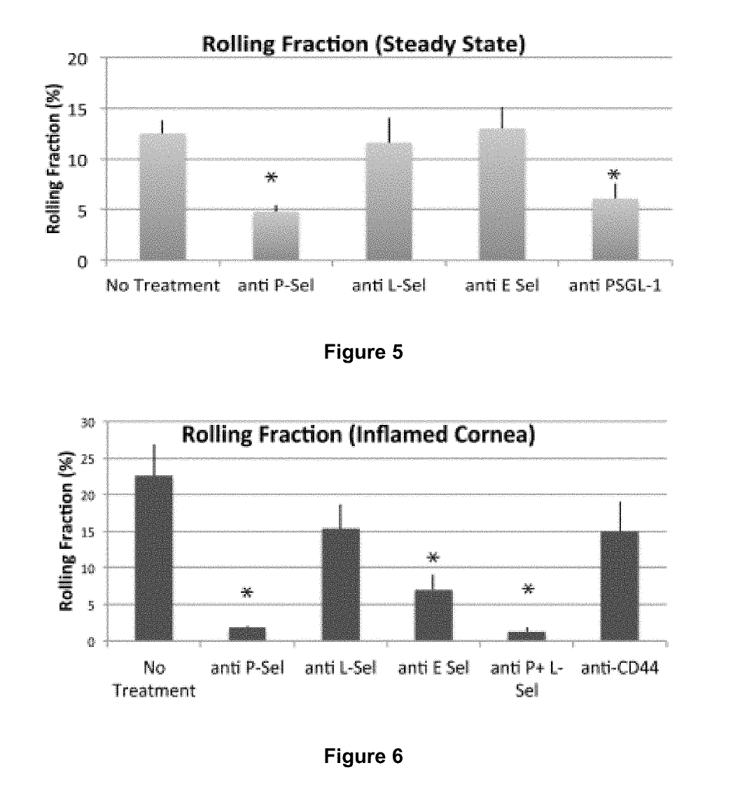

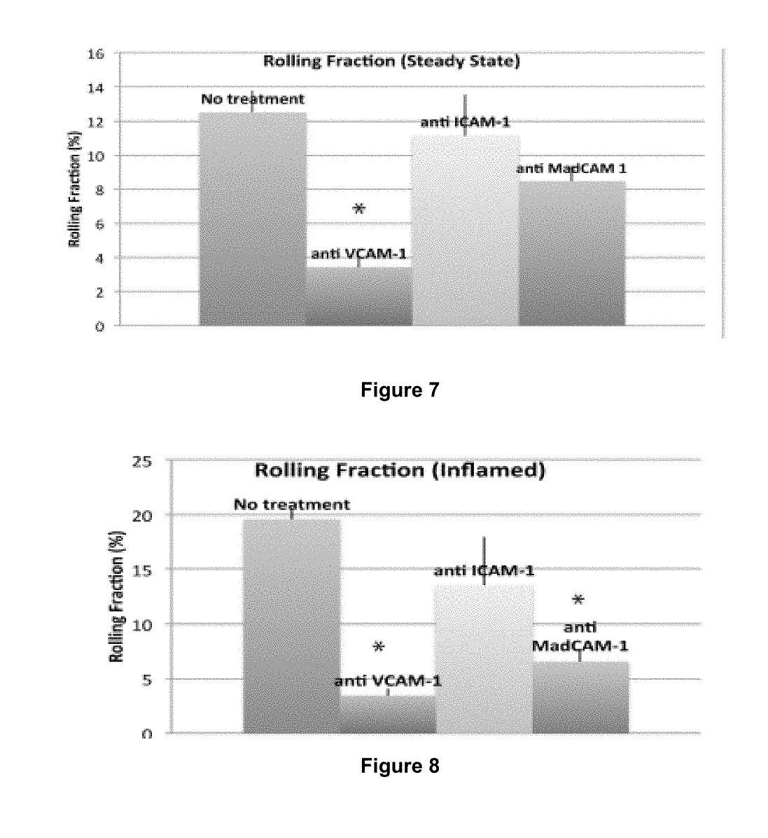

The resulting data show that anti-P-selectin antibodies, anti-PSGL-1 antibodies, and anti-VCAM-1 antibodies significantly decrease the rolling fraction of dendritic cells in the limbal vessel in untreated (steady state) mice (FIGS. 5 and 7). Additional data show that anti-P-selectin antibodies, anti-E-selectin antibodies, a combination of anti-P-selectin and anti-L-selectin antibodies, anti-VCAM-1 antibodies, and anti-MadCAM-1 antibodies significantly decrease the rolling fraction of dendritic cells in the limbal vessel in mice following stimulation of eye inflammation (suture-induced eye inflammation) (FIGS. 6 and 8).

An additional set of experiments was performed to determine the role of different adhesion molecules in dendritic cell sticking in the limbal vessel of the cornea in both untreated (steady state) mice and in mice with suture-induced eye inflammation. In these experiments, the mice were administered the calcein-labelled cells with or without anti-VCAM-1 antibodies, anti-ICAM-1 antibodies, or anti-MadCAM-1 antibodies, thirty minutes before intravital images were obtained.

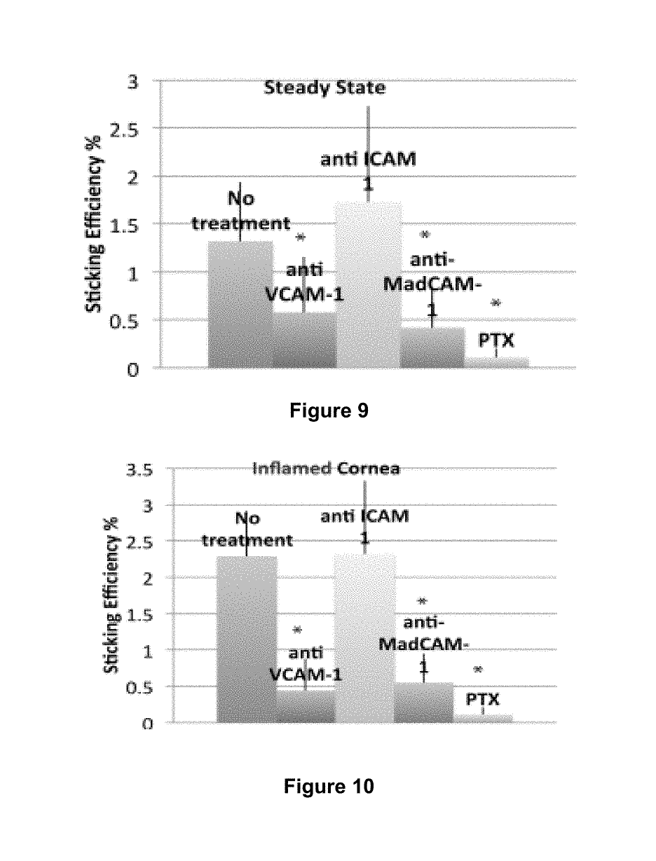

The resulting data show that anti-VCAM-1 antibodies and anti-MadCAM-1 antibodies significantly decrease the sticking efficiency of dendritic cells in the limbal vessel in untreated (steady state) mice, as well as in mice following induction of eye inflammation (suture-induced eye inflammation) (FIGS. 9 and 10, respectively).

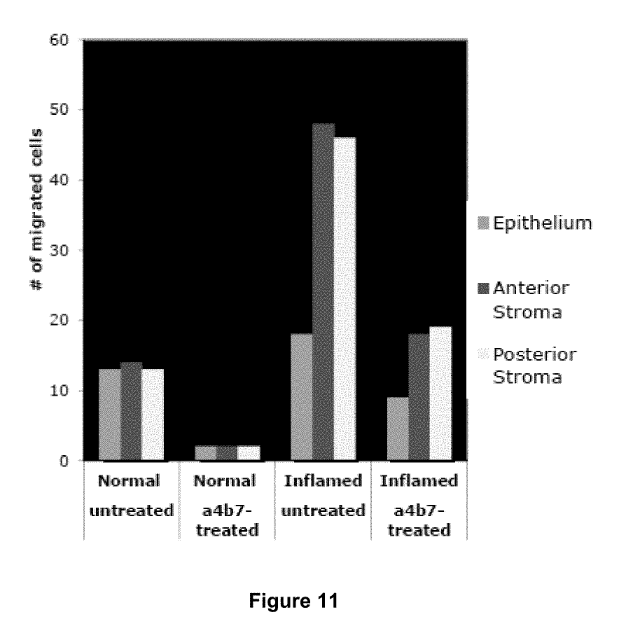

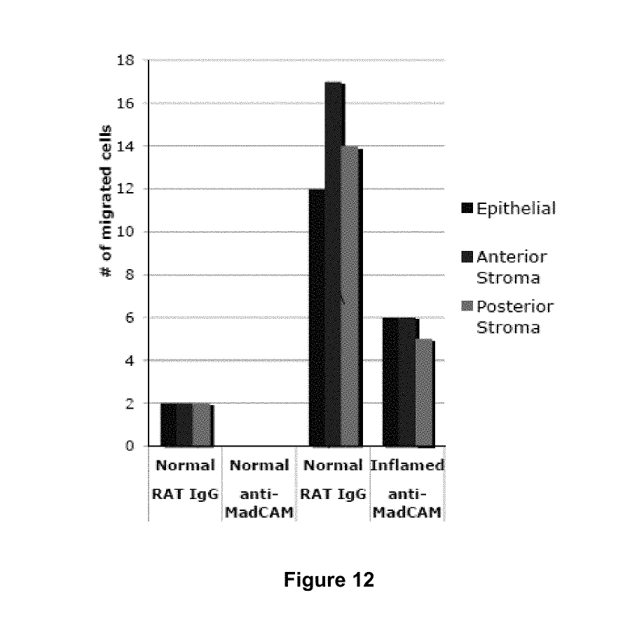

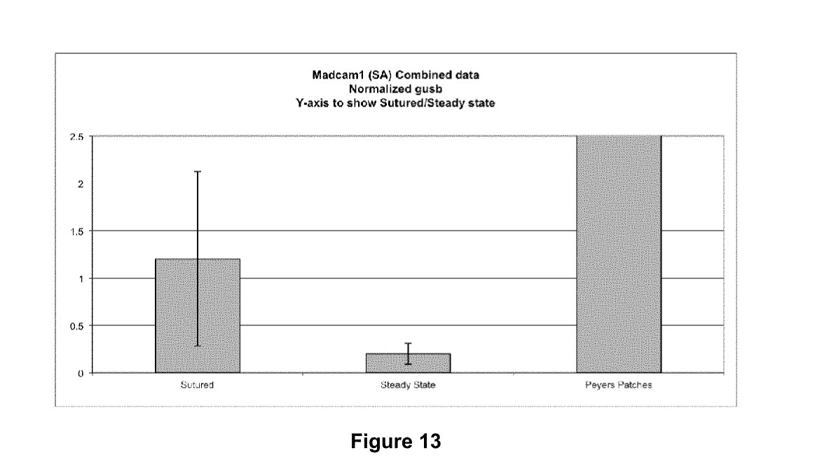

Additional experiments were performed to determine the number of labeled dendritic cells that localized to the corneal epithelium, the corneal anterior stroma, and the corneal posterior stroma in normal (steady state) mice or in mice with suture-induced eye inflammation (inflamed). In these experiments, the two groups of mice were administered calcein-labelled cells treated with a control rat IgG, anti-α4β7 antibodies, or L-selectin antibodies prior to injection, or the mice were administered anti-MadCAM-1 antibodies. In case of anti-α4β7 antibodies the dendritic cells were treated with 10 μL of 0.5 mg/ml antibody for 30 minutes at 4° C., and washed with phosphate buffered saline prior to tail vein injection of the cells to the steady state or inflamed mice. The data show that administration of anti-α4β7 antibodies or administration of anti-MadCAM-1 antibodies significantly decreases dendritic cell migration to the corneal epithelium, the corneal anterior stroma, and the corneal posterior stroma in both untreated (steady state) mice and mice with suture-induced eye inflammation (FIGS. 11 and 12, respectively). These data, for example, indicate that agents that antagonize either α4β7 integrin or MadCAM-1 can be used to reduce dendritic cell migration to the corneal epithelium, the corneal anterior stroma, and the corneal posterior stroma in a subject, can be used to reduce corneal inflammation, and can be used to treat a corneal inflammatory disorder (via their ability to decrease dendritic cell recruitment to the cornea).

RT-PCR was also performed to confirm the expression of MadCAM-1 in the limbal/cornea tissue. The results show that the expression of MadCAM-1 is increased in the limbal/corneal tissue in an inflamed eye, but shows low expression in the limbal/tissue in a normal (steady state) eye.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing corneal inflammation in a subject, the method comprising administering to a subject having corneal inflammation one or more of a MadCAM-1 antagonist, an α4β7 integrin antagonist, a L-selectin antagonist, and an E-selectin antagonist in an amount sufficient to reduce corneal inflammation in the subject.

2. The method of claim 1, wherein the corneal inflammation is chronic corneal inflammation.

3. The method of claim 1, wherein the corneal inflammation is acute corneal inflammation.

4. The method of claim 1, wherein the corneal inflammation is caused by bacterial infection, fungal infection, parasite infection, viral infection, allergies, dry eye disorder, Fuchs' dystrophy, keratoconus, amyloidosis, lattice dystrophy, Stevens Johnson syndrome, physical corneal injury, Behcet's disease, contact lens wear, corneal graft rejection, dry eye syndrome, or immune keratitis.

5. The method of claim 1, further comprising administering to the subject one or more anti-inflammatory agents.

6. A method of reducing dendritic cell recruitment to the cornea, the method comprising administering to a subject having corneal inflammation one or more of a MadCAM-1 antagonist, an α4β7 integrin antagonist, a L-selectin antagonist, and an E-selectin antagonist in an amount sufficient to reduce dendritic cell recruitment to the cornea.

7. The method of claim 6, wherein the dendritic cell recruitment is recruitment of dendritic cells to the corneal epithelium.

8. The method of claim 6, wherein the dendritic cell recruitment is recruitment of dendritic cells to the anterior or posterior stroma of the cornea.

9. The method of claim 6, further comprising selecting a subject having eye inflammation or a corneal inflammatory disorder.

10. The method of claim 6, further comprising administering to the subject one or more anti-inflammatory agents.

11. A method of treating a corneal inflammatory disorder in a subject, the method comprising administering to a subject having a corneal inflammatory disorder one or more of a MadCAM-1 antagonist, an α4β7 integrin antagonist, a L-selectin antagonist, and an E-selectin antagonist in an amount sufficient to decrease corneal inflammation.

12. The method of claim 11, wherein the corneal inflammatory disorder is keratitis.

13. The method of claim 12, wherein the keratitis is non-infectious keratitis.

14. The method of claim 12, wherein the keratitis is infectious keratitis.

15. The method of claim 11, further comprising selecting a subject having a corneal inflammatory disorder.

16. The method of claim 11, further comprising administering to the subject one or more anti-inflammatory agents.

17. The method of claim 11, wherein the MadCAM-1 antagonist is an antibody or an antigen-binding antibody fragment that binds specifically to MadCAM-1.

18. The method of claim 17, wherein the antibody is a fully human antibody or humanized antibody.

19. The method of claim 17, wherein the antigen-binding antibody fragment is selected from the group of: a Fab fragment, a F(ab′)2 fragment, and a scFv fragment.

20. The method of claim 11, wherein the α4β7 integrin antagonist is an antibody or an antigen-binding antibody fragment that binds specifically to α4β7 integrin.

21.-43. (canceled)
